(19) United States
(12) Patent Application Publication
(10) Pub. No.: US 2009/0012071 A1
(43) Pub. Date: Jan. 8, 2009

(54) 4-SUBSTITUTED-1H-ISOTHIAZOLO[5,4-B][1,4]OXAZINO[2,3,4-IJ]QUINOLINE-7,8(2H,9H)-DIONES AND RELATED COMPOUNDS AS ANTI-INFECTIVE AGENTS

(75) Inventors: Barton James Bradbury, Wallingford, CT (US); Milind Deshpande, Madison, CT (US); Akihiro Hashimoto, Branford, CT (US); Ha Young Kim, Cheshire, CT (US); Edlaine Lucien, New Haven, CT (US); Godwin Pais, Hamden, CT (US); Michael John Pucci, Kensington, CT (US); Qiuping Wang, Bethany, CT (US); Jason Allan Wiles, Hamden, CT (US)

Correspondence Address: CANTOR COLBURN, LLP, 20 Church Street, 22nd Floor, Hartford, CT, 06103, US

(73) Assignee: ACHILLION PHARMACEUTICALS, INC., New Haven, CT (US)

(21) Appl. No.: 12/108,106

(22) Filed: Apr. 23, 2008

Related U.S. Application Data
(60) Provisional application No. 60/913,404, filed on Apr. 23, 2007.

(51) Int. Cl. A61K 31/5383; C07D 498/06; A61P 33/02; A61P 31/04

(52) U.S. Cl. 514/229.5; 544/99

(57) ABSTRACT

The present invention provides compounds of the formula that possess antimicrobial activity. Certain compounds provided herein possess potent antibacterial, antiprotozoal, or antifungal activity. Particular compounds provided herein are also potent and/or selective inhibitors of microbial DNA synthesis and reproduction. The invention provides anti-microbial compositions, including pharmaceutical compositions, containing a compound of the invention and one or more or more carriers. The invention provides pharmaceutical compositions containing a 4-substituted-1H-isothiazolo[5,4-b][1,4]oxazino[2,3,4-ij]quinoline-7,8(2H,9H)-dione or related compound as the only active agent or in combination with one or more other active agents. The invention provides methods for treating or preventing microbial infections, preferably animals, by administering an effective amount of a 4-substituted-1H-isothiazolo[5,4-b][1,4]oxazino[2,3,4-ij]quinoline-7,8(2H,9H)-dione or related compound to an organism suffering from or susceptible to microbial infection. The invention also provides methods of inhibiting microbial growth and survival by applying an effective amount of a 4-substituted-1H-isothiazolo[5,4-b][1,4]oxazino[2,3,4-ij]quinoline-7,8(2H,9H)-dione or related compound.

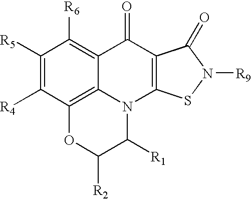

### CROSS REFERENCE TO RELATED APPLICATION

This application claims priority from U.S. Provisional Application No. 60/913,404, filed Apr. 23, 2007, which is hereby incorporated in its entirety.

### FIELD OF THE INVENTION

The present invention provides 4-substituted-1H-isothiazolo[5,4-b][1,4]oxazino[2,3,4-ij]quinoline-7,8(2H,9H)-diones and related compounds, which possess antimicrobial activity. Certain compounds provided herein possess potent antibacterial, antiprotozoal, or antifungal activity. Particular compounds provided herein are also potent and/or selective inhibitors of microbial DNA synthesis and reproduction. The invention provides anti-microbial compositions, including pharmaceutical compositions, containing a compound of the invention and one or more carriers. The invention provides pharmaceutical compositions containing a 4-substituted-1H-isothiazolo[5,4-b][1,4]oxazino[2,3,4-ij]quinoline-7,8(2H,9H)-dione or related compound as the only active agent or in combination with one or more other active agents. The invention provides methods for treating or preventing microbial infections, by providing an effective amount of a 4-substituted-1H-isothiazolo[5,4-b][1,4]oxazino[2,3,4-ij]quinoline-7,8(2H,9H)-dione or related compound to a patient suffering from or susceptible to microbial infection.

### BACKGROUND OF THE INVENTION

Antimicrobial compounds are compounds capable of destroying or suppressing the growth or reproduction of microorganisms, such as bacteria, protozoa, mycoplasma, yeast, and fungi. The mechanisms by which antimicrobial compounds act vary. However, they are generally believed to function in one or more of the following ways: by inhibiting cell wall synthesis or repair; by altering cell wall permeability; by inhibiting protein synthesis; or by inhibiting synthesis of nucleic acids. For example, beta-lactam antibacterials inhibit the essential penicillin binding proteins (PBPs) in bacteria, which are responsible for cell wall synthesis. Quinolones act, at least in part, by inhibiting DNA synthesis, thus preventing the cell from replicating.

Many attempts to produce improved antimicrobials yield equivocal results. Indeed, few antimicrobials are produced that are truly clinically acceptable in terms of their spectrum of antimicrobial activity, avoidance of microbial resistance, and pharmacology. Thus there is a continuing need for broad-spectrum antimicrobials, and a particular need for antimicrobials effective against resistant microbes.

Pathogenic bacteria are known to acquire resistance via several distinct mechanisms including inactivation of the antibiotic by bacterial enzymes (e.g., beta-lactamases that hydrolyze penicillin and cephalosporins); removal of the antibiotic using efflux pumps; modification of the target of the antibiotic via mutation and genetic recombination (e.g., penicillin-resistance in Neiserria gonorrhea); and acquisition of a readily transferable gene from an external source to create a resistant target (e.g., methicillin-resistance in Staphylococcus aureus or MRSA). There are certain gram-positive pathogens, such as vancomycin-resistant Enterococcus faecium, which are resistant to virtually all commercially available antibiotics.

Resistant organisms of particular note include methicillin-resistant and vancomycin-resistant Staphylococcus aureus, penicillin-resistant Streptococcus pneumoniae, vancomycin-resistant enterococci, fluoroquinolone-resistant E. coli, cephalosporin-resistant aerobic gram-negative rods and imipenem-resistant Pseudomonas aeruginosa. These organisms are significant causes of nosocomial infections and are clearly associated with increasing morbidity and mortality. The increasing numbers of elderly and immunocompromised patients are particularly at risk for infection with these pathogens. Therefore, there is a large unmet medical need for the development of new antimicrobial agents.

### SUMMARY OF THE INVENTION

The inventors have discovered compounds having a 1H-isothiazolo[5,4-b][1,4]oxazino[2,3,4-ij]quinoline-7,8(2H,9H)-dione core that are unexpectedly potent inhibitors of bacterial replication and survival. Certain of these compounds are particularly potent inhibitors of Methicillan resistant S. Aureus replication and survival. Without wishing to be bound to any particular theory, the inventors believe that the unexpected improvement in potency is due to certain novel 4-position substituents on the 1H-isothiazolo[5,4-b][1,4]oxazino[2,3,4-ij]quinoline-7,8(2H,9H)-dione core. Additionally compounds described herein possess superior metabolic stability.

The invention provides compounds of Formula I and Formula II (shown below) and includes 4-substituted-1H-isothiazolo[5,4-b][1,4]oxazino[2,3,4-ij]quinoline-7,8(2H,9H)-diones and related compounds, which possess antimicrobial activity. The invention provides compounds of Formula I and Formula II that possess potent and/or selective antibacterial, antiprotozoal, or antifungal activity. The invention also provides anti-bacterial compositions containing one or more compounds of Formula I or Formula II, or a salt, solvate, or acylated prodrug of such a compound, and one or more pharmaceutically acceptable carriers.

The invention further comprises methods of treating and preventing microbial infections, particularly bacterial and protozoal infections by providing an effective amount of a compound of Formula I or Formula II to a patient having or susceptible to a microbial infection. These microbial infections include bacterial infections, for example E. coli infections, Staphylococcus infections, Salmonella infections and protozoal infections, for example chlamydia infections. The invention includes methods of preventing or treating microbial infections in mammalian patients, including humans, but also encompasses methods of preventing or treating microbial infections in other animals, including fish, birds, reptiles, and amphibians.

Methods of treatment include administering a compound of Formula I or Formula II alone as the single active agent or administering a compound of Formula I or Formula II in combination with one or more other therapeutic agent, such as an antibacterial, an antifungal, an antiviral, an interferon, an efflux-pump inhibitor, a beta-lactamase inhibitor, or another compound of Formula I or Formula II.

The invention also provides methods of inhibiting microbial growth and survival by applying an effective amount of a 4-substituted-1H-isothiazolo[5,4-b][1,4]oxazino[2,3,4-ij]quinoline-7,8(2H,9H)-dione or related compound. The invention includes, for example, methods of inhibiting microbial growth and survival on medical instruments or on surfaces used for food preparation by applying a composition containing a compound of Formula I or Formula II.

A compound or pharmaceutically acceptable salt of Formula I, or its tautomer of Formula II:

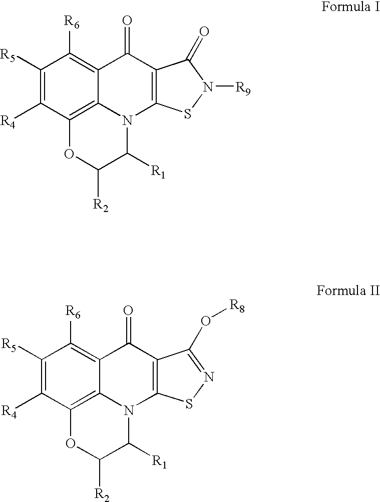

Within Formula I and Formula II the following definitions apply.

R₁ and R₂ are independently hydrogen, halogen, amino, ═CH₂, C₁-C₄alkyl, C₂-C₄alkenyl, C₁-C₄alkoxy, mono- or di-C₁-C₄alkylamino, C₁-C₂haloalkyl, or C₁-C₂haloalkoxy.

R₄ is a nitrogen-linked heterocycloalkyl group, which has 4 to 8 ring members, including 0, 1, or 2 additional ring heteroatoms independently chosen from N, O, and S, substituted with 0, 1, or more substituents (a), 0 or 1 substituent (b), and 1 or 2 substituents (c), or

R₄ is a nitrogen-linked C₁-C₄alkylamino substituted with a 5- or 6-membered heteroaryl group having 1 or 2 heteroatoms independently chosen from N, O, and S, or substituted with a heterocycloalkyl group, which has 4 to 8 ring members, including 1 or 2 ring heteroatoms independently chosen from N, O, and S; substituted with 0, 1, or more substituents (a), 0 or 1 substituent (b), and 0 or 1 substituent (c), or

R₄ is a nitrogen-linked heterocycloalkyl or heterocycloalkenyl group, each of which has 4 to 8 ring members, including 0, 1, or 2 additional ring heteroatoms independently chosen from N, O, and S, forming part of a bicyclic system with a 3- to 8-membered cycloalkyl or heterocycloalkyl ring in fused or spiro orientation, substituted with 0, 1, or more substituents (a), 0 or 1 substituent (b), and 0 or 1 substituent (c), or

R₄ is a nitrogen-linked 6-membered heterocycloalkyl group, 0, 1, or 2 additional ring heteroatoms independently chosen from N, O, and S, and bridged with a methylene or ethylene bridge, substituted with 0, 1, or more substituents (a), 0 or 1 substituent (b), and 1 substituent (c).

Within this definition of R₄:

(a) is halogen, hydroxy, amino, nitro, —C(O)NH₂, C₁-C₄alkyl, C₁-C₄alkoxy, C₁-C₂haloalkyl, or C₁-C₂haloalkoxy,

(b) is hydroxyC₁-C₄alkyl, aminoC₁-C₄alkyl, mono- or di-(C₁-C₄alkyl)amino, mono- or di-alkylcarboxamide, or phenyl, each of which is unsubstituted, and

(c) is oxo, cyano, branched C₁-C₄alkyl, C₁-C₆alkylthio, C₁-C₆alkoxy, C₂-C₆alkanoyl, (C₃-C₇cycloalkyl)C₀-C₄alkyl, C₁-C₆alkylester, or an amino group of the formula

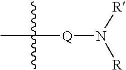

where Q is absent or C₁-C₄alkyl, R is hydrogen or C₁-C₄alkyl, and R′ is (C₃-C₇cycloalkyl)(C₀-C₄alkyl), (5- or 6-membered heterocycloalkyl)C₀-C₄alkyl, or (aryl)C₀-C₄alkyl.

Each of (c) other than oxo and cyano is substituted with 0, 1, or 2 substituents independently chosen from halogen, hydroxy, amino, cyano, nitro, oxo, C₁-C₂alkyl, C₁-C₂alkoxy, C₁-C₂hydroxyalkyl, C₁-C₂-aminoalkyl, or mono- or di-(C₁-C₂alkyl)amino.

Or, R₄ is a group of the formula:

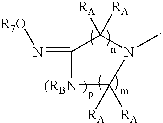

Where, R₇ is hydrogen, or R₇ is C₁-C₈alkyl, C₂-C₆alkenyl, C₂-C₆alkynyl, C₂-C₆alkanoyl, (C₃-C₇cycloalkyl)C₀-C₄carbohydryl, (C₄-C₇cycloalkenyl)C₀-C₄carbohydryl, (aryl)C₀-C₄carbohydryl, (aryl)(C═O)—, mono- or di-(C₁-C₆alkyl)carboxamide, C₁-C₆alkylester, or (heterocycloalkyl)C₀-C₄carbohydryl, each of which is substituted with 0 to 5 substituents independently chosen from halogen, hydroxy, amino, cyano, nitro, C₁-C₄alkyl, C₁-C₄alkoxy, C₁-C₂haloalkyl, C₁-C₂haloalkoxy, mono- and di-C₁-C₄alkylamino, C₂-C₄alkanoyl, C₁-C₄alkylthio, —O(C═O)R₁₀, —(C═O)NR₁₀R₁₁, —O(C═O)NR₁₀R₁₁, —(C═O)OR₁₀,—(C═O)NR₁₀OR₁₁, —NR₁₀(C═O)R₁₁, —NR₁₀(C═O)OR₁₁, —NR₁₀(C═O)NR₁₁R₁₂, —NR₁₀(C═S)NR₁₁R₁₂, —NR₁₀NR₁₁R₁₂, —SO₃R₁₀, —(S═O)OR₁₀, —SO₂R₁₃, —SO₂NR₁₀R₁₁, or —NR₁₀SO₂R₁₃.

The variable m is 1, 2, or 3; n is 1, 2, or 3; and p is 0 or 1.

Each R_{A} is independently (i), (ii), or (iii); where

(i) is hydrogen, hydroxy, amino, or cyano;

(ii) is C₁-C₆alkyl, C₂-C₆alkenyl, C₂-C₆alkynyl, C₁-C₆alkoxyC₀-C₄alkyl, mono- or di-C₁-C₄alkylamino, C₁-C₂haloalkoxy, (C₃-C₇cycloalkyl)C₀-C₄-carbohydryl, (C₄-C₇cycloalkenyl)C₀-C₄-carbohydryl, (aryl)C₀-C₆-carbohydryl, (aryl)C₁-C₄alkoxy, (heterocycloalkyl)C₀-C₄-carbohydryl, (heteroaryl)C₀-C₆-carbohydryl, C₁-C₆alkylthio, —(C₀-C₄alkyl)O(C═O)R₁₀, —(C₀-C₄alkyl)(C═O)NR₁₀R₁₁, —(C₀-C₄alkyl)O(C═O)NR₁₀R₁₁, —(C₀-C₄alkyl)(C═O)OR₁₀, —(C₀-C₄alkyl)NR₁₀(C═O)R₁₁, —(C₀-C₄alkyl)NR₁₀(C═O)OR₁₁, —(C₀-C₄alkyl)NR₁₀(C═O)NR₁₁R₁₂, —(C₀-C₄alkyl)NR₁₀(C═S)NR₁₁R₁₂, —(C₀-C₄alkyl)NR₁₀NR₁₁R₁₂, —(C₀-C₄alkyl)N═NR₁₃, —(C₀-C₄alkyl)SO₃R₁₀, —(C₀-C₄alkyl)(S═O)OR₁₀, —(C₀-C₄alkyl)SO₂R₁₃, —(C₀-C₄alkyl)SO₂NR₁₀R₁₁, —(C₀-C₄alkyl)NR₁₀SO₂R₁₃, ═N—OH, or ═N—O(C₀-C₄alkyl);

(iii) is —OR_{D}, —(C═O)R_{D}, —SO₂R_{D}, —SO₃R_{D}, or —NR₁₀SO₂R_{D}, where R_{D} is C₁-C₄alkyl, (C₃-C₇cycloalkyl)C₀-C₂alkyl, (heterocycloalkyl)C₀-C₂alkyl, (aryl)C₀-C₂alkyl, or (heteroaryl)C₀-C₂alkyl; where each of (ii) and (iii) is substituted with 0 to 3 substituents independently chosen from halogen, hydroxy, amino, cyano, nitro, —COOH, —(C═O)OCH₃, —CONH₂, C₁-C₄alkyl, C₂-C₄alkenyl, C₂-C₄alkynyl, C₁-C₄alkoxy, (C₃-C₇cycloalkyl)C₀-C₄-carbohydryl, (C₃-C₇cycloalkyl)C₀-C₄alkoxy, mono- and di-C₁-C₄alkylamino, C₁-C₂haloalkyl, C₁-C₂haloalkoxy, and C₂-C₄alkanoyl.

Or, any two R_{A} bound to the same carbon atom may be joined to form a C₃-C₇cycloalkyl, or a 3- to 7-membered heterocycloalkyl group, having 1 or 2 heteroatoms independently chosen from N, O, and S; each of which 3- to 7-membered heterocycloalkyl group is substituted with 0 to 2 substituents independently chosen from C₁-C₂alkyl and C₁-C₂alkoxy.

Or, any two R_{A} bound to different carbon atoms may be joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl and C₁-C₂alkoxy.

R_{B} is hydrogen or C₁-C₄alkyl;

R₅ is hydrogen, halogen, hydroxy, amino, cyano, C₁-C₄alkyl, C₁-C₄alkoxy, or mono- or di-(C₁-C₄)alkylamino.

R₆ is hydrogen, halogen, hydroxy, amino, cyano, nitro, —NH₂, C₁-C₄alkyl, C₁-C₄alkoxy, mono- or di-(C₁-C₄)alkylamino, mono-, di- or tri-C₁-C₄ alkylhydrazinyl, C₂-C₄alkanoyl, C₁-C₄alkylester, C₁-C₂haloalkyl, or C₁-C₂haloalkoxy.

R₈ is hydrogen, C₁-C₆alkyl, or C₂-C₆alkanoyl.

R₉ is hydrogen, C₁-C₄alkyl, (C₃-C₇cycloalkyl)C₀-C₂alkyl, or (phenyl)C₀-C₂alkyl, each of which is substituted with 0 to 3 substituents independently chosen from halogen, hydroxy, amino, C₁-C₂alkyl, C₁-C₂alkoxy, mono- and di-(C₁-C₂)alkylamino, C₁-C₂haloalkyl, and C₁-C₂haloalkoxy.

### DETAILED DESCRIPTION OF THE INVENTION

### Chemical Description and Terminology

Prior to setting forth the invention in detail, it may be helpful to provide definitions of certain terms to be used herein. Compounds of the present invention are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

An “active agent” means a compound (including a compound of Formula I or II), element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound, crystalline forms, non-crystalline forms, and any polymorphs of the compound are included.

Compounds of Formula I or II may contain one or more asymmetric elements such as stereogenic centers, including chiral centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present invention. Formula I includes all chiral forms, stereoisomers, diastereomers, and enantiomers of compounds of Formula I.

The term “chiral” refers to molecules, which have the property of non-superimposability of the mirror image partner.

“Stereoisomers” are compounds, which have identical chemical constitution, but differ with regard to the arrangement of the atoms or groups in space.

A “Diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

Where a compound exists in various tautomeric forms, the invention is not limited to any one of the specific tautomers, but rather includes all tautomeric forms.

The invention includes compounds of Formula I and II having all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula that includes variables, e.g. R₁, R₂, R₄, R₅, R₆, R₈, and R₉. Unless otherwise specified, each variable within Formulas I and II is defined independently of other variables. Thus, if a group is said to be substituted, e.g. with 0-2 R*, then said group may be substituted with up to two R* groups and R* at each occurrence is selected independently from the definition of R*. Also, combinations of substituents and/or variables are permissible only if such combinations result in stable compounds.

The term “substituted,” as used herein, means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent. For example, —(CH₂)C₃-C₇cycloalkyl is attached through carbon of the methylene (CH₂) group.

A “bridged” group such as a bridged methylene or ethylene is a divalent radical covalently bound at each end to non-adjacent ring carbon atoms of a carbocycle or heterocycle.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1 to about 6 carbon atoms. When C₀-CN alkyl is used herein in conjunction with another group, for example, (phenyl)C₀-C₄alkyl, the indicated group, in this case phenyl, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, 3-methylbutyl, t-butyl, n-pentyl, and sec-pentyl.

“Alkyl-O—N═” is an alkylimino group. The alkyl group is as defined above and is covalently bound to the oxygen of an imino group. The alkyl-O—N═ is attached to the group it substitutes via a covalent double bond to the imino nitrogen atom.

“Alkanoyl” is an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

“Alkenyl” means straight and branched hydrocarbon chains comprising one or more unsaturated carbon-carbon double bonds, which may occur in any stable point along the chain. Alkenyl groups described herein typically have from 2 to about 12 carbons atoms. Preferred alkenyl groups are lower alkenyl groups, those alkenyl groups having from 2 to about 8 carbon atoms, e.g. C₂-C₈, C₂-C₆, and C₂-C₄ alkenyl groups. Examples of alkenyl groups include ethenyl, propenyl, and butenyl groups.

“Alkynyl” means straight and branched hydrocarbon chains comprising one or more unsaturated carbon-carbon triple bonds, which may occur in any stable point along the chain. Alkenyl groups described herein typically have from 2 to about 12 carbons atoms. Preferred alkynyl groups are lower alkynyl groups, those alktnyl groups having from 2 to about 8 carbon atoms, e.g. C₂-C₈, C₂-C₆, and C₂-C₄ alkynyl groups.

“Alkoxy” means an alkyl group, as defined above, with the indicated number of carbon atoms attached to the group it substitutes via an oxygen bridge, thus alkyl-O—. Examples of alkoxy include, but are not limited to, methoxy, ethoxy, 3-hexoxy, and 3-methylpentoxy.

“Mono- and/or di-alkylamino” indicates secondary or tertiary alkyl amino groups, wherein the alkyl groups are as defined above and have the indicated number of carbon atoms. The alkylamino group is bound to the group it substitutes at the nitrogen atom. The alkyl groups or the mono- and/or di-alkylamino are independently chosen. Examples of mono- and di-alkylamino groups include ethylamino, dimethylamino, and methyl-propyl-amino. “Mono- and/or dialkylaminoalkyl” groups are mono- and/or di-alkylamino groups attached through an alkyl linker having the specified number of carbon atoms, for example a di-methylaminoethyl group. Tertiary amino substituents may by designated by nomenclature of the form N—R—N—R′, indicating that the groups R and R′ are both attached to a single nitrogen atom.

“Alkylester” indicates an alkyl group as defined above attached through an ester linkage. The ester linkage may be in either orientation, e.g. a group of the formula —O(C═O)alkyl or a group of the formula —(C═O)O-alkyl.

“Aryl” means aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl. In the terms (Aryl)alkyl and (aryl)carbohydryl, aryl, alkyl, and carbohydryl are as defined herein and the point of attachment to the substituted group is in the alkyl or carbohydryl chain.

“Mono- and/or di-alkylcarboxamide” indicates groups of formula (alkyl₁)-NH—(C═O)— and (alkyl)(alkyl₂)—N—(C═O)— in which the alkyl and alkyl₂ groups are independently chosen alkyl groups as defined above having the indicated number of carbon atoms. Mono and/or di-alkylcarboxamide also refers to groups of the formula (alkyl)(C═O)—NH— and (alkyl)(C═O)(alkyl₂)N—, carboxamide groups in which the point of attachment is the nitrogen atom, in which the alkyl and alkyl₂ groups are independently chosen alkyl groups as defined above having the indicated number of carbon atoms.

The term “alkylthio” indicates an alkyl group as defined above attached through a sulfur linkage, i.e. a group of the formula alkyl-S—. Examples include ethylthio and pentylthio.

As used herein, the term “aminoalkyl” indicates an alkyl group as defined above substituted with at least one amino substituent. Similarly, the term “hydroxyalkyl” indicates an alkyl group as defined above, substituted with at least one hydroxyl substituent. In certain instances the alkyl group of the aminoalkyl or hydroxyalkyl group may be further substituted.

“Carbohydryl” as used herein, includes both branched and straight-chain hydrocarbon groups, which are saturated or unsaturated, having the specified number of carbon atoms. Examples of carbohydryl groups include C₁-C₆alkyl groups, such as methyl, or 5-butyl, C₂-C₆alkynyl groups such as hexynyl, and C₂-C₆ alkenyl groups, such as 1-propenyl. When C₀-Cₙ carbohydryl is used herein in conjunction with another group, for example, (C₃-C₇cycloalkyl)C₀-C₄-carbohydryl, the indicated group, in this case C₃-C₇cycloalkyl, is either directly bound by a single covalent bond (C₀), or attached by a carbohydryl chain having the specified number of carbon atoms, in this case from 1 to about 4 carbon atoms.

“Cycloalkyl” indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane.

“Cycloalkenyl” as used herein, indicates an unsaturated, but not aromatic, hydrocarbon ring having at least one ring carbon-carbon double bond. Cycloalkenyl groups contain from 4 to about 8 carbon atoms, usually from 4 to about 7 carbon atoms. Examples include cyclohexenyl and cyclobutenyl.

“Heteroaryl” indicates a stable 5- to 7-membered monocyclic or 7- to 10-membered bicyclic heterocyclic ring which contains at least 1 aromatic ring that contains from 1 to 4, or preferably from 1 to 3, heteroatoms chosen from N, O, and S, with remaining ring atoms being carbon. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the heteroaryl group is not more than 1. A nitrogen atom in a heteroaryl group may optionally be quaternized. When indicated, such heteroaryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a [1,3]dioxolo[4,5-c]pyridyl group. In some instances 5- or 6-membered heteroaryl groups are indicated. Such groups are always monocyclic heteroaryl groups. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, and 5,6,7,8-tetrahydroisoquinoline.

“Heterocycloalkyl” means a saturated cyclic group containing from 1 to about 3 heteroatoms chosen from N, O, and S, with remaining ring atoms being carbon. Heterocycloalkyl groups have from 3 to about 8 ring atoms, and more typically have from 5 to 7 ring atoms. Examples of heterocycloalkyl groups include morpholinyl, piperazinyl, piperidinyl, and pyrrolidinyl groups. A nitrogen in a heterocycloalkyl group may optionally be quaternized.

“Heterocycloalkenyl” as used herein, indicates an unsaturated, but not aromatic, heterocyclic ring having at least one ring double bond. A heterocycloalkenyl group contains from 1 to 3 heteroatoms chosen from N, O, and S, with remaining ring atoms being carbon. Cycloalkenyl groups contain from 4 to about 8 carbon atoms, usually from 4 to about 7 carbon atoms. Examples include cyclohexenyl and cyclobutenyl.

“Haloalkyl” indicates both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge (oxygen of an alcohol radical).

“Halo” or “halogen” as used herein refers to fluoro, chloro, bromo, or iodo. The term “mono-, di-, or tri-alkylhydrazinyl” indicates from 1 to 3 independently chosen alkyl groups as defined above attached through a single-bonded nitrogen-nitrogen linkage. At least one of the alkyl groups is attached to the terminal nitrogen (the nitrogen not bound to the core structure). When the term mono- or di-alkylhydrazinyl is used only the terminal nitrogen is alkyl substituted. Examples of alkylhydrazinyl groups include 2-butyl-1-hydrazinyl, 2-butyl-2-methyl-1-hydrazinyl, and 1,2-dimethyl-2-propyl-1-hydrazinyl.

“Pharmaceutical compositions” are compositions comprising at least one active agent, such as a compound or salt of Formula I, and at least one other substance, such as a carrier, excipient, or diluent. Pharmaceutical compositions meet the U.S. FDA's GMP (good manufacturing practice) standards for human or non-human drugs.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions of the invention refers to a diluent, excipient, or vehicle with which an active compound is administered.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

A “patient” is a human or non-human animal in need of medical treatment. Medical treatment can include treatment of an existing condition, such as a disease or disorder, prophylactic or preventative treatment, or diagnostic treatment. In some embodiments the patient is a human patient.

“Prodrug” means any compound that becomes compound of the invention when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of the invention.

“Providing” means giving, administering, selling, distributing, transferring (for profit or not), manufacturing, compounding, or dispensing.

“Providing a compound of Formula I or II with at least one other active agent” means the compound of Formula I or II and the other active agent(s) are provided simultaneously in a single dosage form, provided concomitantly in separate dosage forms, or provided in separate dosage forms for administration separated by some amount of time that is within the time in which both the compound of Formula I or II and the at least one other active agent are within the blood stream of a patient. The compound of Formula I or II and the other active agent need not be prescribed for a patient by the same medical care worker. The additional active agent or agents need not require a prescription. Administration of the compound of Formula I or II or the at least one additional active agent can occur via any appropriate route, for example, oral tablets, oral capsules, oral liquids, inhalation, injection, suppositories or topical contact.

“Treatment,” as used herein includes providing an amount of a compound of Formula I or II sufficient to: (a) prevent a disease or a symptom of a disease from occurring in a patient who may be predisposed to the disease but has not yet been diagnosed as having it (e.g. including diseases that may be associated with or caused by a primary disease; (b) inhibiting the disease, i.e. arresting its development; and (c) relieving the disease, i.e., causing regression of the disease.

A “therapeutically effective amount” of a pharmaceutical combination of this invention means an amount effective, when administered to a patient, to provide a therapeutic benefit such as an amelioration of symptoms, e.g., an amount effective to decrease the symptoms of a microbial infection. For example, a therapeutically effective amount can be an amount effective to produce a significant reduction in the detectable level of microbial pathogens in a patient's blood or bodily fluids. A “significant reduction” is an amount that is statistically significant in a standard parametric test of statistical significance such as Student's T-test, where p<0.05.

### Antimicrobial Compounds

In addition to the compounds of Formula I and Formula II, described above the invention also includes compounds of Formula I and Formula II in which the variables (e.g. R₁, R₂, R₄, R₅, etc.) carry definitions other than those set forth above.

For example the invention includes compounds of Formula I and II, wherein the variables R₁—R₉ are defined as follows.

R₁ and R₂ are independently hydrogen, halogen, amino, ═CH₂, C₁-C₄alkyl, C₂-C₄alkenyl, C₁-C₄alkoxy, mono- or di-C₁-C₄alkylamino, C₁-C₂haloalkyl, or C₁-C₂haloalkoxy.

R₄ is a nitrogen-linked heterocycloalkyl group, which has 5 or 6 ring members, including 0 or 1 additional ring heteroatoms independently chosen from N, O, and S, substituted with 0, 1, or more substituents (a), 0 or 1 substituent (b), and 1 or 2 substituents (c); where

(a) is halogen, hydroxy, amino, nitro, —C(O)NH₂, C₁-C₄alkyl, C₁-C₄alkoxy, C₁-C₂haloalkyl, or C₁-C₂haloalkoxy,

(b) is hydroxyC₁-C₄alkyl, aminoC₁-C₄alkyl, mono- or di-(C₁-C₄alkyl)amino, mono- or di-alkylcarboxamide, or phenyl, each of which is unsubstituted, and

(c) is oxo, cyano, HO—N═, C₁-C₄alkyl-O—N═, branched C₁-C₄alkyl, C₁-C₆alkylthio, C₁-C₆alkoxy, C₂-C₆alkanoyl, (C₃-C₇cycloalkyl)C₀-C₄alkyl, C₁-C₆alkylester, or an amino group of the formula or an amino group of the formula

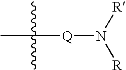

where Q is absent or C₁-C₄alkyl, R is hydrogen or C₁-C₄alkyl, and R′ is (C₃-C₇cycloalkyl)(C₀-C₄alkyl), (5- or 6-membered heterocycloalkyl)C₀-C₄alkyl, or (aryl)C₀-C₄alkyl.

Each (c) other than oxo, cyano, and HO—N═ is substituted with 0, 1, or 2 substituents independently chosen from halogen, hydroxy, amino, cyano, nitro, oxo, C₁-C₂alkyl, C₁-C₂alkoxy, C₁-C₂hydroxyalkyl, C₁-C₂-aminoalkyl, or mono- or di-(C₁-C₂alkyl)amino.

R₅ is hydrogen, halogen, hydroxy, amino, cyano, C₁-C₄alkyl, C₁-C₄alkoxy, or mono- or di-(C₁-C₄)alkylamino.

R₆ is hydrogen, halogen, hydroxy, amino, cyano, nitro, —NHNH₂, C₁-C₄alkyl, C₁-C₄alkoxy, mono- or di-(C₁-C₄)alkylamino, mono-, di- or tri-C₁-C₄ alkylhydrazinyl, C₂-C₄alkanoyl, C₁-C₄alkylester, C₁-C₂haloalkyl, or C₁-C₂haloalkoxy.

R₈ is hydrogen, C₁-C₆alkyl, or C₂-C₆alkanoyl.

R₉ is hydrogen, C₁-C₄alkyl, (C₃-C₇cycloalkyl)C₀-C₂alkyl, or (phenyl)C₀-C₂alkyl, each of which is substituted with 0 to 3 substituents independently chosen from halogen, hydroxy, amino, C₁-C₂alkyl, C₁-C₂alkoxy, mono- and di-(C₁-C₂)alkylamino, C₁-C₂haloalkyl, and C₁-C₂haloalkoxy.

Other alternate definitions for the variables shown in Formula I and Formula II follow. The invention includes embodiments in which various definitions set forth below for the variables are combined. All combinations of the following definitions are encompassed within the scope of the invention so long the combination results in a stable compound.

### The R₁ and R₂ Variables:

(1) R₁ is hydrogen or methyl; and R₂ is hydrogen, ═CH₂, or C₁-C₄alkyl.

(2) R₁ is hydrogen.

(3) R₁ is C₁-C₄alkyl.

(4) R₁ is hydrogen or C₁-C₃alkyl; and R₂ is hydrogen, ═CH₂, or C₁-C₄alkyl.

(5) R₁ is methyl and R₂ is hydrogen.

### The R₄ Variable:

(1) R₄ is a nitrogen-linked heterocycloalkyl group, which has 4 to 8 ring members, including 0, 1, or 2 additional ring heteroatoms independently chosen from N, O, and S, substituted with 0, 1, or more substituents (a), 0 or 1 substituent (b), and 1 or 2 substituents (c).

(2) R₄ is a nitrogen-linked heterocycloalkyl group, which has 4 to 8 ring members, including 0, 1, or 2 additional ring heteroatoms independently chosen from N, O, and S, substituted with 0, 1, or more substituents (a), 0 or 1 substituent (b), and 1 or 2 substituents (c). Wherein

(a) is halogen, hydroxy, amino, unsubstituted C₁-C₂alkyl, trifluoromethyl, or trifluoromethoxy;

(b) is hydroxyC₁-C₂alkyl or aminoC₁-C₂alkyl, and

(c) is oxo, cyano, C₁-C₆alkylthio, C₁-C₆alkoxy, C₂-C₆alkanoyl, (C₃-C₇cycloalkyl)C₀-C₄alkyl, C₁-C₆alkylester, or an amino group of the formula

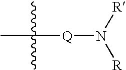

where Q is absent or C₁-C₄alkyl, R is hydrogen or C₁-C₄alkyl, and R′ is (C₃-C₇cycloalkyl)(C₀-C₄alkyl), (5- or 6-membered heterocycloalkyl)C₀-C₄alkyl; or (phenyl)C₀-C₄alkyl; each of which (c) other than oxo and cyano is substituted with 0 to 2 substituents independently chosen from halogen, hydroxy, amino, C₁-C₂alkyl, C₁-C₂alkoxy, and mono- and di-(C₁-C₂)alkylamino.

In certain of these embodiments the nitrogen-linked heterocycloalkyl group is a pyrrolidinyl, piperidinyl, piperazinyl, morpholinyl, or thiomorpholinyl group.

(3) R₄ is a pyrrolidinyl, piperidinyl, piperazinyl, morpholinyl, or thiomorpholinyl group; with 0, 1, or more substituents (a), 0 or 1 substituent (b), and 1 or 2 substituents (c).

Within this embodiment:

(a) is halogen, hydroxy, amino, unsubstituted C₁-C₂alkyl, trifluoromethyl, or trifluoromethoxy;

(b) is hydroxyC₁-C₂alkyl or aminoC₁-C₂alkyl, and

(c) is oxo, C₁-C₄alkylthio, C₁-C₄alkoxy, C₂-C₄alkanoyl, or (C₃-C₇cycloalkyl)C₀-C₂alkyl, or an amino group of the formula

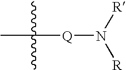

where Q is absent or C₁-C₂alkyl, R is hydrogen or C₁-C₄alkyl, and R′ is (C₃-C₇cycloalkyl)(C₀-C₂alkyl) or (phenyl)C₀-C₄alkyl; each of which (c) other than oxo is substituted with 0 to 2 substituents independently chosen from halogen, hydroxy, amino, C₁-C₂alkyl, C₁-C₂alkoxy, and mono- and di-(C₁-C₂)alkylamino.

(4) R₄ is a group of the formula:

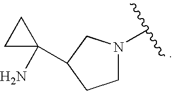

(5) R₄ is a nitrogen-linked C₁-C₄alkylamino substituted with a 5 or 6-membered heteroaryl group having 1 or 2 heteroatoms independently chosen from N, O, and S, or R₄ is a nitrogen-linked C₁-C₄alkylamino substituted with a heterocycloalkyl group, which has 4 to 8 ring members, including 1 or 2 ring heteroatoms independently chosen from N, O, and S; each of which R₄ is substituted with 0, 1, or more substituents (a), 0 or 1 substituent (b), and 0 or 1 substituent (c).

(6) R₄ is a nitrogen-linked C₁-C₄alkylamino substituted with a pyridyl, pyrimidinyl, pyrrolyl, imidazolyl, piperazinyl, piperidinyl, morphonlinyl, pyrrolidinyl, or oxazolyl group, each of which is substituted with 0, 1, or more substituents (a), 0 or 1 substituent (b), and 0 or 1 substituent (c).

(7) R₄ is a nitrogen-linked C₁-C₄alkylamino substituted with a 5 or 6-membered heteroaryl group, in which the 5- or 6-membered heteroaryl is substituted with 0 or 1 or more substituents independently chosen from halogen, hydroxy, amino, C₁-C₂alkyl, C₁-C₂alkoxy, C₁-C₂haloalkyl, C₁-C₂haloalkoxy, hydroxyC₁-C₂alkyl, aminoC₁-C₂alkyl, and mono- and di-(C₁-C₂alkyl)amino.

(8) R₄ is a nitrogen-linked heterocycloalkyl or heterocycloalkenyl group, each of which has 4 to 8 ring members, including 0, 1, or 2 additional ring heteroatoms independently chosen from N, O, and S, forming part of a bicyclic system with a 3- to 8-membered cycloalkyl or heterocycloalkyl ring in fused or spiro orientation, substituted with 0, 1, or more substituents (a), 0 or 1 substituent (b), and 0 or 1 substituent (c).

(9) R₄ is a nitrogen-linked pyrrolidinyl group fused to a 6-membered cycloalkyl group or fused to a 6-membered heterocycloalkyl group containing one nitrogen atom.

Or, R₄ is an nitrogen-linked pyrrolidinyl group forming part of a bicyclic system with a 3- to 5-membered cycloalkyl ring in spiro orientation or forming part of a bicyclic system with a 3- to 5-membered heterocycloalkyl ring, containing one ring nitrogen atom, in spiro orientation.

Or, R₄ is a nitrogen-linked piperidinyl group forming part of a bicyclic system with a 3- to 5-membered cycloalkyl ring in spiro orientation or a 3- to 5-membered heterocycloalkyl ring, containing either one ring nitrogen atom or 1 or 2 oxygen atoms, in spiro orientation.

Within this embodiment R₄ is substituted with 0 or 1 or more substituents independently chosen from halogen, hydroxy, amino, C₁-C₂alkyl, C₁-C₂alkoxy, C₁-C₂haloalkyl, C₁-C₂haloalkoxy, hydroxyC₁-C₂alkyl, aminoC₁-C₂alkyl, and mono- and di-(C₁-C₂alkyl)amino.

(10) R₄ is a nitrogen-linked 6-membered heterocycloalkyl group, 0, 1, or 2 additional ring heteroatoms independently chosen from N, O, and S, and bridged with a methylene or ethylene bridge, substituted with 0, 1, or more substituents (a), 0 or 1 substituent (b), and 1 substituent (c).

(11) R₄ is a methylene bridged piperidinyl group or a methylene bridged piperazinyl group; substituted with 0 or 1 or more substituents independently chosen from halogen, hydroxy, amino, C₁-C₂alkyl, and C₁-C₂alkoxy.

(12) R₄ is a group of the Formula I:

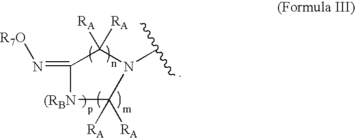

(13) In certain embodiments in which R₄ is a group of Formula III,

R₇ is hydrogen, or

R₇ is C₁-C₈alkyl, C₂-C₆alkanoyl, (C₃-C₇cycloalkyl)C₀-C₄-carbohydryl, (aryl)C₀-C₄-carbohydryl, (aryl)(C═O)—, mono- or di-(C₁-C₆alkyl)carboxamide, C₁-C₆alkylester, or (heterocycloalkyl)C₀-C₄-carbohydryl, each of which is substituted with 0 to 5 substituents independently chosen from halogen, hydroxy, amino, cyano, nitro, C₁-C₄alkyl, C₁-C₄alkoxy, C₁-C₂haloalkyl, C₁-C₂haloalkoxy, mono- and di-C₁-C₄alkylamino, C₂-C₄alkanoyl, C₁-C₄alkylthio, —O(C═O)R₁₀, —(C═O)NR₁₀R₁₁, —O(C═O)NR₁₀R₁₁, —(C═O)OR₁₀, —(C═O)NR₁₀R₁₁, —NR₁₀(C═O)R₁₁, —NR₁₀(C═O)OR₁₁, —NR₁₀(C═O)NR₁₁R₁₂, —NR₁₀(C═S)NR₁₁R₁₂, —NR₁₀NR₁₁R₁₂, —SO₃R₁₀, —(S═O)OR₁₀, —SO₂R₁₃, —SO₂NR₁₀R₁₁, or —NR₁₀SO₂R₁₃.

(14) In other embodiments in which R₄ is a group of Formula III, R₇ is hydrogen, or R₇ is C₁-C₈alkyl, (C₃-C₇cycloalkyl)C₀-C₄-carbohydryl, (aryl)C₀-C₄-carbohydryl, or (heterocycloalkyl)C₀-C₄-carbohydryl, each of which is substituted with 0 to 5 substituents independently chosen from halogen, hydroxy, amino, cyano, C₁-C₄alkyl, C₁-C₄alkoxy, C₁-C₂haloalkyl, C₁-C₂haloalkoxy, mono- and di-C₁-C₄alkylamino, C₂-C₄alkanoyl, —(C═O)NR₁₀R₁₁, and —NR₁₀(C═O)R₁₁.

(15) In other embodiments in which R₄ is a group of Formula III, R₇ is hydrogen, or R₇ is C₁-C₈alkyl or (aryl)C₀-C₄alkyl, each of which is substituted with 0 to 5 substituents independently chosen from halogen, hydroxy, amino, cyano, C₁-C₄alkyl, C₁-C₄alkoxy, C₁-C₂haloalkyl, C₁-C₂haloalkoxy, mono- and di-C₁-C₄alkylamino, C₂-C₄alkanoyl, —(C═O)NR₁₀R₁₁, and —NR₁₀(C═O)R₁₁.

(16) In still other embodiments in which R₄ is a group of Formula III, R₇ is hydrogen, C₁-C₂alkyl, or benzyl.

(17) In certain embodiments in which R₄ is a group of Formula III, n is 1; m is 2, p is 1 and R_{B} is hydrogen.

(18) In certain other embodiments in which R₄ is a group of Formula III, n is 1; m is 2, and p is 0.

(19) In still other embodiments in which R₄ is a group of Formula III, n is 2, m is 2, and p is 0.

(20) In any embodiment in which R₄ is a group of Formula III, R_{A} may carry the following definition.

Each R_{A} is independently (i), (ii), or (iii); where

(i) is hydrogen, hydroxy, amino, or cyano;

(ii) is C₁-C₆alkyl, C₂-C₆alkenyl, C₂-C₆alkynyl, (C₁-C₆alkoxy)C₀-C₄alkyl, mono- or di-C₁-C₄alkylamino, C₁-C₂haloalkoxy, (C₃-C₇cycloalkyl)C₀-C₄-carbohydryl, (C₄-C₇cycloalkenyl)C₀-C₄-carbohydryl, (aryl)C₀-C₆-carbohydryl, (aryl)C₁-C₄alkoxy, (heterocycloalkyl)C₀-C₄-carbohydryl, (heteroaryl)C₀-C₆-carbohydryl, C₁-C₆alkylthio, —(C₀-C₄alkyl)(C═O)NR₁₀R₁₁, —(C₀-C₄alkyl)NR₁₀(C═O)R₁₁, ═N—OH, or ═N—O(C₀-C₄alkyl); and

(iii) is —OR_{D} or —(C═O)R_{D}, where R_{D} is C₁-C₄alkyl, (C₃-C₇cycloalkyl)C₀-C₂alkyl, (heterocycloalkyl)C₀-C₂alkyl, (aryl)C₀-C₂alkyl, or (heteroaryl)C₀-C₂alkyl.

Each of which (ii) and (iii) is substituted with 0 to 3 substituents independently chosen from halogen, hydroxy, amino, cyano, nitro, —COOH, —(C═O)OCH₃, —CONH₂, C₁-C₄alkyl, C₂-C₄alkenyl, C₂-C₄alkynyl, C₁-C₄alkoxy, (C₃-C₇cycloalkyl)C₀-C₄-carbohydryl, (C₃-C₇cycloalkyl)C₀-C₄alkoxy, mono- and di-C₁-C₄alkylamino, C₁-C₂haloalkyl, C₁-C₂haloalkoxy, and C₂-C₄alkanoyl; or

any two R_{A} bound to the same carbon atom may be joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl and C₁-C₂alkoxy; or

any two R_{A} bound to different carbon atoms may be joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl and C₁-C₂alkoxy.

(21) In any additional embodiment in which R₄ is a group of Formula III, R_{A} may carry the following definition.

Each R_{A} is independently (i) or (ii); where:

(i) is hydrogen or amino; and

(ii) is C₁-C₆alkyl, (C₁-C₆alkoxy)C₀-C₄alkyl, mono- or di-C₁-C₄alkylamino, (C₃-C₇cycloalkyl)C₀-C₄-carbohydryl, (aryl)C₀-C₆-carbohydryl, (heterocycloalkyl)C₀-C₄-carbohydryl, (heteroaryl)C₀-C₆-carbohydryl, —(C₀-C₄alkyl)(C═O)NR₁₀R₁₁, —(C₀-C₄alkyl)NR₁₀(C═O)R₁₁, ═N—OH, or ═N—O(C₀-C₄alkyl).

Within this definition of R_{A} each of (ii) is substituted with 0 to 3 substituents independently chosen from halogen, hydroxy, amino, cyano, nitro, —COOH, —(C═O)OCH₃, —CONH₂, C₁-C₄alkyl, C₂-C₄alkenyl, C₂-C₄alkynyl, C₁-C₄alkoxy, (C₃-C₇cycloalkyl)C₀-C₄-carbohydryl, (C₃-C₇cycloalkyl)C₀-C₄alkoxy, mono- and di-C₁-C₄alkylamino, C₁-C₂haloalkyl, C₁-C₂haloalkoxy, and C₂-C₄alkanoyl; or

any two R_{A} bound to the same carbon atom may be joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl, and C₁-C₂alkoxy; or

any two R_{A} bound to different carbon atoms may be joined to form a C₃-C₇cycloalkyl or heterocycloalkyl group having 1 or 2 heteroatoms independently chosen from N, O, and S; each of which is substituted with 0 to 2 substituents independently chosen from C₁-C₂alkyl and C₁-C₂alkoxy.

(22) In any other embodiment in which R₄ is a group of Formula III, R_{A} may carry the following definition. Each R_{A} is independently (i) or (ii); where

(i) is hydrogen or amino; and

(ii) is C₁-C₆alkyl, mono- or di-C₁-C₄alkylamino, (C₃-C₇cycloalkyl)C₀-C₄alkyl, (heterocycloalkyl)C₀-C₄alkyl, —(C₀-C₄alkyl)(C═O)NR₁₀R₁₁, —(C₀-C₄alkyl)NR₁₀(C═O)R₁₁, ═N—OH, or ═N—O(C₀-C₄alkyl).

Within this definition each of (ii) is substituted with 0 to 3 substituents independently chosen from hydroxy, amino, cyano, and —CONH₂; and any two R_{A} bound to the same carbon atom may be joined to form a C₃-C₇cycloalkyl or heterocycloalkyl group having 1 or 2 heteroatoms independently chosen from N, O, and S; each of which is substituted with 0 to 2 substituents independently chosen from C₁-C₂alkyl and C₁-C₂alkoxy.

(23) In any other embodiment in which R₄ is a group of Formula III, R_{A} may carry the following definition. Each R_{A} is independently (i) or (ii); where (i) is hydrogen or amino, and (ii) is C₁-C₆alkyl, (C₃-C₇cycloalkyl)C₀-C₂alkyl, or mono- or di-C₁-C₄alkylamino,

where each of (ii) is substituted with 0 to 3 amino.

Within this definition any two R_{A} bound to the same carbon atom may be joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl and C₁-C₂alkoxy; or any two R_{A} bound to different carbon atoms may be joined to form a C₃-C₇cycloalkyl or a membered heterocycloalkyl group having 1 or 2 heteroatoms independently chosen from N, O, and S; each of which is substituted with 0 to 2 substituents independently chosen from C₁-C₂alkyl and C₁-C₂alkoxy.

(24) In any additional embodiment in which R₄ is a group of Formula III, R_{A} may have the following definition: R_{A} is independently hydrogen, C₁-C₄alkyl or C₁-C₄alkyl substituted with one amino substituent.

(25) In other embodiments in which R₄ is a group of Formula III, R_{A} may carry the following definition: one or two R_{A} is C₁-C₄alkyl substituted with one amino substituent and the remaining R_{A} are hydrogen.

(26) R_{A} may also carry the following definition:

Two R_{A} bound to the same carbon atom are joined to form a C₃-C₇cycloalkyl or a 3- to 7-membered heterocycloalkyl group having 1 or 2 heteroatoms independently chosen from N, O, and S; each of which is substituted with 0 to 2 substituents independently chosen from C₁-C₂alkyl and C₁-C₂alkoxy; and the remaining R_{A} are independently chosen from hydrogen, C₁-C₂alkyl and C₁-C₂alkoxy.

(27) In other embodiments R_{A} carries the following definition:

Two R_{A} bound to the same carbon atom are joined to form a C₃-C₄cycloalkyl or a 3- to 4-membered heterocycloalkyl group having 1 N atom; each of which is substituted with 0 to 2 substituents independently chosen from C₁-C₂alkyl and C₁-C₂alkoxy; and the remaining R_{A} are independently chosen from hydrogen, C₁-C₂alkyl, and C₁-C₂alkoxy.

(28) In other embodiments R₄ is a group of Formula IV, where R_{A} and R₇ carry any of the definitions set forth herein for these variables.

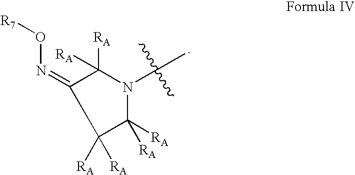

(29) The invention includes embodiments in which

R₄ is a nitrogen-linked heterocycloalkyl group, in which the N-linked heterocycloalkyl group is a pyrrolidinyl, piperidinyl, piperazinyl, morpholinyl, or thiomorpholinyl group substituted with 0, 1, or more substituents (a) and 1 substituent (c); where

(a) is halogen, hydroxy, amino, —C(O)NH₂, C₁-C₂alkyl, C₁-C₂alkoxy, trifluoromethyl, or trifluoromethoxy, and

(c) is oxo, cyano, HO—N═, C₁-C₄alkyl-O—N═, branched C₁-C₄alkyl, C₁-C₄alkoxy, or (C₃-C₇cycloalkyl)C₀-C₄alkyl, wherein each of (c) other than oxo, cyano, and HO—N═ is substituted with 0, 1, or 2 substituents independently chosen from halogen, hydroxy, amino, oxo, C₁-C₂alkoxy, C₁-C₂-aminoalkyl, or mono- or di-(C₁-C₂alkyl)amino.

In certain of these embodiments

(a) is halogen, amino, C₁-C₂alkyl, C₁-C₂alkoxy, and

(c) is HO—N═, C₁-C₂alkyl-O—N═, branched C₁-C₄alkyl, or (cyclopropyl)C₀-C₂alkyl; wherein the branched C₁-C₄alkyl and the (cyclopropyl)C₀-C₂alkyl are substituted with 0 or 1 halogen, hydroxy, amino, or mono- or di-(C₁-C₂alkyl)amino substituent.

(30) R₄ is a group of the formula:

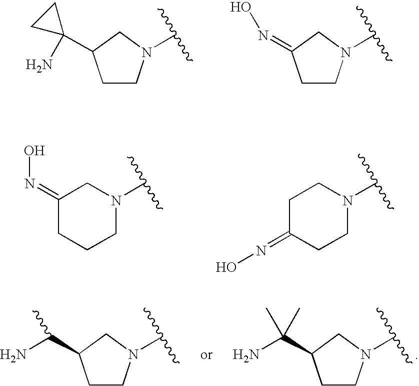

### The R₅ Variable:

(1) R₅ is hydrogen, halogen, or amino.

(2) R₅ is hydrogen, halogen, or amino.

(3) R₅ is fluoro.

(4) R₅ is halogen.

### The R₆ Variable:

(1) R₆ is hydrogen, amino, mono- or di-(C₁-C₂)alkylamino, or mono- or di-C₁-C₂ alkylhydrazinyl.

(2) R₆ is hydrogen.

(3) R₆ is hydrogen, amino, C₁-C₂alkyl, mono- or di-(C₁-C₂)alkylamino.

The R₈ and R₉ Variables:

(1) R₈ is hydrogen or C₁-C₄alkyl.

(2) R₉ is hydrogen, C₁-C₄alkyl, (C₃-C₇cycloalkyl)C₀-C₂alkyl, or (phenyl)C₀-C₂alkyl.

(3) R₈ is hydrogen or methyl.

(4) R₉ is hydrogen.

(5) The compound is a compound of Formula II and R₈ is hydrogen or the compound is a compound of Formula I and R₉ is hydrogen.

(6) The compound is a compound of Formula I and R₉ is C₁-C₄alkyl, (C₃-C₇cycloalkyl)C₀-C₂alkyl, or (phenyl)C₀-C₂alkyl.

Certain compounds of Formula I and Formula II possess potent antibacterial, antifungal, and/or antiprotozoal activity. Particular compounds of the invention exhibit Minimum Inhibitory Concentrations (MIC) of 64 μg/ml or less against Staphyloccocus aureus and/or Escherichia coli in a standard assay for determining the MIC of a compound against these bacteria, such as the assay provided in Example 5 below. Preferred compounds of the Formula I and II exhibit MIC values of 10 μg/ml or less against Staphyloccocus aureus and/or Escherichia coli. More preferred compound of the Formula I and II exhibit MIC values of 4 μg/ml or less, or even more preferably 1 μg/ml or less, against Staphyloccocus aureus and/or Escherichia coli.

Certain compounds of Formula I and Formula II are selective antimicrobial agents; having the ability to kill or inhibit the growth or reproduction of microbial organisms, while having little or no effect on the cells of fish, amphibians, reptiles, birds, or mammals. The selectivity of compounds of Formula I and Formula II may be assessed by determining the CC₅₀ (the concentration at which 50% of the cells are killed) for cultured cells of a higher animal, such as a fish, reptiles, amphibian, bird, or mammal. Certain compounds of the invention exhibit a CC₅₀ of greater than 100 micromolar for mammalian cells. Certain compounds of the invention exhibit a CC₅₀ of greater than 100 micromolar for cultured human hepatocytes, and also exhibit MIC values of 641 g/ml or less, preferably 10 μg/ml or less, or more preferably 4 μg/ml or less, or still more preferably 1 μg/ml or less against Staphyloccocus aureus and/or Escherichia coli.

Without wishing to be bound to any particular theory it is believed that the antimicrobial properties of compounds of Formula I and Formula II are due to the ability of these compounds to inhibit the activity of microbial DNA gyrases while having little or no effect on the analogous enzyme, Topoisomerase II, present in higher organisms. Certain preferred compound of the invention are 100-fold or more selective for bacterial DNA gyrases than for mammalian, particularly human, Topoisomerase II.

### Pharmaceutical Preparations

Compounds and salts of Formula I and Formula II can be administered as the neat chemical, but are preferably administered as a pharmaceutical composition or formulation. Accordingly, the invention provides pharmaceutical compositions comprising a compound or pharmaceutically acceptable salt of Formula I or Formula II, together with one or more pharmaceutically acceptable carrier, excipients, adjuvant, diluent, or other ingredient.

Compounds of general Formula I and II may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

Binders are substances that bind or “glue” powders together and make them cohesive by forming granules, thus serving as the “adhesive” in the formulation. Binders add cohesive strength already available in the diluent or bulking agent Examples of binders include starch, gelatin, natural sugars, corn sweeteners, natural and synthetic gums such as acacia, sodium alginate, carboxymethylcellulose, polyethylene glycol and waxes. The amount of binder in the composition can range, for example, from about 2 to about 20% by weight of the composition, or from about 3 to about 10% by weight, even more preferably from about 3 to about 6% by weight.

Diluents include sugars such as lactose, sucrose, mannitol and sorbitol; starches derived from wheat, corn, rice and potato; and celluloses such as microcrystalline cellulose. The amount of diluent in the composition may be, for example, about 10 to about 90% by weight of the total composition, about 25 to about 75%, about 30 to about 60% by weight, or about 12 to about 60%.

Disintegrants are materials added to a pharmaceutical composition to help it break apart (disintegrate) and release the active agent. Suitable disintegrants include starches; including “cold water soluble” modified starches such as sodium carboxymethyl starch; natural and synthetic gums such as locust bean, karaya, guar, and tragacanth gum and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range, for example, from about 2 to about 15% by weight of the composition or from about 4 to about 10% by weight.

Lubricants are substances added to a pharmaceutical composition to enable the tablet, granules, etc. after it has been compressed, to release from the; mold or die by reducing friction or wear. Examples of lubricants useful in pharmaceutical dosage forms include boric acid, sodium benzoate, sodium acetate, sodium chloride, and the like. Lubricants are usually added at the very last step before tablet compression, since they must be present on the surfaces of the granules and in between them and the parts of the tablet press. The amount of lubricant in the composition can range, for example, from about 0.2 to about 5% by weight of the composition, from about 0.5 to about 2%, or from about 0.3 to about 1.5% by weight. Amount of compound or salt of Formula I or II in a unit dose may be generally varied or adjusted from about 1.0 milligram to about 1,000 milligrams, from about 1.0 to about 900 milligrams, from about 1.0 to about 500 milligrams, or from about 1 to about 250 milligrams, according to the particular application and the potency of the compound. The actual dosage employed may be varied depending upon the patient's age, sex, weight and severity of the condition being treated.

Pharmaceutical compositions formulated for oral administration are often preferred. These compositions contain between 0.1 and 99% of a compound of the invention and usually at least about 5% (weight %) of a compound of the invention. Some embodiments contain from about 25% to about 50% or from 5% to 75% of a compound of invention.

### Liquids Formulations

Compounds of the invention can be incorporated into oral liquid preparations such as aqueous or oily suspensions, solutions, emulsions, syrups, tinctures, syrups, or elixirs, for example. Moreover, formulations containing these compounds can be presented as a dry product, e.g. as granules or powders, for constitution with water or other suitable vehicle before use. Typical components of carriers for syrups, elixirs, emulsions and suspensions include ethanol, glycerol, propylene glycol, polyethylene glycol, liquid sucrose, sorbitol and water. Liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid). Oral formulations may contain demulcent, flavoring agents, sweetening agents, such as sucrose or saccharin, taste-masking agents, and coloring agents.

### Suspensions

Aqueous suspensions contain the active material(s) in admixture with excipients suitable for the manufacture of aqueous suspensions. Such excipients are suspending agents, for example Avicel RC-591, sodium carboxymethylcellulose, methylcellulose, hydropropylmethylcellulose, sodium alginate, polyvinylpyrrolidone, gum tragacanth and gum acacia; dispersing or wetting agents, for example lecithin and polysorbate 80. The aqueous suspensions may also contain one or more preservatives, for example ethyl, n-propyl p-hydroxybenzoate, methyl parabens, propyl parabens, and sodium benzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

### Emulsions

Pharmaceutical compositions of the invention may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

### Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture.

Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard or soft shell capsules. A capsule is a dosage form administered in a special container or enclosure containing an active agent. The active agent may be present in solid, liquid, gel, or powder form, or any other pharmaceutically acceptable form. A capsule shell may be made of methyl cellulose, polyvinyl alcohols, or denatured gelatins or starch or other material. Hard shell capsules are typically made of blends of relatively high gel strength bone and pork skin gelatins. Soft shell capsule shells are often made of animal or plant gelatins. The capsule itself may contain small amounts of dyes, opaquing agents, plasticizers and preservatives.

### Injectable and Parenteral Formulations

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid are useful in the preparation of injectables.

Compounds of Formula I and II may be administered parenterally in a sterile medium. Parenteral administration includes subcutaneous injections, intravenous, intramuscular, intrathecal injection or infusion techniques. The drug, depending on the vehicle and concentration used, can either be suspended or dissolved in the vehicle. Advantageously, adjuvants such as local anesthetics, preservatives and buffering agents can be dissolved in the vehicle. In compositions for parenteral administration the carrier typically comprises least about 90% by weight of the total composition.

### Packaged Formulations

The invention includes packaged pharmaceutical compositions. Packaged formulations include a compound or salt of Formula I or Formula II in a container and instructions for using the compound to treat an animal (typically a human patient) suffering from a microorganism infection) or to prevent a microorganism infection in an animal. For example, the instructions may be prescribing information, provided to a patient or health care provider, or a label in a packaged pharmaceutical composition. Prescribing information may include, for example, efficacy, dosage and administration, contraindication and adverse reaction information pertaining to the pharmaceutical composition.

The instructions may be instructions for using the compositions to treat a bacterial, mycoplasm, or protozoal infection. For example the instructions may be instructions for using the composition to treat a urinary or genital tract infection, such as pyelonephritis, cervical gonococcal infections, cystitis, urethral chlamydial infections, cervical chlamydial infections, urethral gonococcal infections, and prostatitis, a respiratory infection, such as lower respiratory tract infections, acute sinusitis, acute exacerbations of chronic bronchitis, community-acquired pneumonia, and nosocomial pneumonia, skin infections, such as skin-structure infections, impetigo, folliculitis, boils, scalded skin syndrome, and cellulites, and other infections such as bone infections, joint infections, infectious diarrhea, typhoid fever, intra-abdominal infections, gynecologic infections, including toxic shock syndrome, pelvic infections, and post-surgical infections. The instructions may be instructions for using the composition to treat a patient suffering from a bacterial infection, such as a S. aureus infection.

In all of the foregoing the compounds of the invention can be administered alone or as mixtures, and the compositions may further include additional drugs or excipients as appropriate for the indication.

### Methods of Treatment

The invention includes methods of preventing and treating microorganism infections, particularly bacterial and protozoal infections, by administering an effective amount of one or more compounds of Formula I and of Formula II to an animal at risk for a microorganism infection or suffering from a microorganism infection. The animal may be a fish, amphibian, reptile or bird, but is preferably a mammal. Methods of treating and preventing microorganism infections in livestock animals, companion animals, and human patients are particularly preferred.

The compounds disclosed herein are useful for preventing and treating bacterial infections in animals. Furthermore compounds of the invention may be used to treat a variety of conditions not attributed to bacterial infections. These include diseases and disorders caused fungal infections, mycoplasma infections, protozoal infections, or other conditions involving infectious organisms.

In some circumstances an effective amount of a compound of Formula I or Formula II may be an amount sufficient to reduce the symptoms of the microorganism infection. Alternatively an effective amount of a Compound of Formula I may be an amount sufficient to significantly reduce the amount of microorganism or antibodies against the detectable in a patient's tissues or bodily fluids.

Methods of treatment also include inhibiting microorganism replication in vivo, in an animal at risk for a microorganism infection or suffering from such an infection, by administering a sufficient concentration of a compound of Formula I or Formula II to inhibit bacterial survival in vitro. By “sufficient concentration” of a compound administered to the patient is meant the concentration of the compound available in the animal's system to prevent or combat the infection. Such a concentration by be ascertained experimentally, for example by assaying blood concentration of the compound, or theoretically, by calculating bioavailability. The amount of a compound sufficient to inhibit bacterial survival in vitro may be determined with a conventional assay for bacterial survival such as the Minimum Inhibitory Concentration (MIC) Assay disclosed in Example 3, which follows.

The invention also includes using compounds of Formula I and Formula II in prophylactic therapies. In the context of prophylactic or preventative treatment an effective amount of a compound of the invention is an amount sufficient to significantly decrease the treated animal's risk of contracting a microorganism infection.

Compounds of the invention are particularly useful for treating and preventing infectious disorders. These include for example: ocular infections such as conjunctivitis; urinary tract and genital infections, such as complicated urinary tract infections, acute urinary tract and genital infections, such as pyelonephritis, cervical gonococcal infections, cystitis, urethral chlamydial infections, cervical chlamydial infections, urethral gonococcal infections, and prostatitis, respiratory infections, such as lower respiratory tract infections, acute sinusitis, acute exacerbations of chronic bronchitis, community-acquired pneumonia, and nosocomial pneumonia, skin infections, such as skin-structure infections, impetigo, folliculitis, boils, scalded skin syndrome, and cellulites, and other infections such as bone infections, joint infections, infectious diarrhea, typhoid fever, intra-abdominal infections, gynecologic infections, including toxic shock syndrome, pelvic infections, and post-surgical infections.

The disclosed compounds are useful for treating infections caused by the following microorganisms:

Aerobic Gram-positive Microorganisms: Including but not limited to Enterococcus faecalis, Enterococcus faecium, Staphylococcus aureus (including methicillan S. aureus), Staphylococcus epidermidis, Staphylococcus saprophyticus, Streptococcus pneumoniae, Streptococcus pyogenes, Staphylococcus haemolyticus, and Staphylococcus hominis.

Aerobic Gram-negative Microorganisms: Including but not limited to Campylobacter jejuni, Citrobacter diversus, Citrobacter freundii, Enterobacter cloacae, Escherichia coli, Haemophilus influenzae, Haemophilus parainfluenzae, Klebsiella pneumoniae, Moraxella catarrhalis, Morganella morganii, Neisseria gonorrhoeae, Proteus mirabilis, Proteus vulgaris, Providencia rettgeri, Providencia stuartii, Pseudomonas aeruginosa, Stenotrophomonas maltophila, Salmonella typhi, Serratia marcescens, Shigella boydii, Shigella dysenteriae, Shigella flexneri, Shigella sonnei. Acinetobacter Iwoffi, Aeromonas hydrophila, Edwardsiella tarda, Enterobacter aerogenes, Klebsiella oxytoca, Legionella pneumophila, Pasteurella multocida, Salmonella enteritidis, Vibrio cholerae, Vibrio parahaemolyticus, Vibrio vulnificus, Yersinia enterocolitica and H. Pylori.

Non-bacterial microorganisms: Mycoplasma, Legionella and Chlamydia.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### Combination Administration

The compounds of the invention may also be useful in combination with other pharmaceutically active agents such as antibacterial agents, antiviral agents, antifungal agents, anti-inflammatories, interferon, efflux-pump inhibitors, and beta-lactamase inhibitors. Antibiotic agents include any molecule that tends to prevent, inhibit or destroy life and as such, includes anti-bacterial agents, anti-fungicides, anti-viral agents, and anti-parasitic agents.

Pharmaceutical compositions of the invention include single dosage forms containing of a compound of Formula I and/or Formula II and one or more other active agent, dosage forms containing more than one compound of Formula I and/or Formula II, and separate administration of a compound of Formula I and/or Formula II with another active agent.

The following active agents, which are useful in combinations of the invention, may be isolated from an organism that produces the agent or synthesized by methods known to those of ordinary skill in the art of medicinal chemistry or purchased from a commercial source.

Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones. Examples of antibiotic agents include, but are not limited to, Penicillin G (CAS Registry No.: 61-33-6); Methicillin (CAS Registry No.: 61-32-5); Nafcillin 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefmetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

Anti-fungals agents include but are not limited to Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

Antiviral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, ZaIcitabine, Zidovudine, Acemannan, Acetylleucine, Amantadine, Amidinomycin, Delavirdine, Foscarnet, Indinavir, Interferon-α, Interferon-β, Interferon-γ, Kethoxal, Lysozyme, Methisazone, Moroxydine, Nevirapine, Podophyllotoxin, Ribavirin, Rimantadine, Ritonavir, Saquinavir, Stailimycin, Statolon, Tromantadine, and Xenazoic Acid.

Antiinflammatory agents include, but are not limited to, Enfenamic Acid, Etofenamate, Flufenamic Acid, Isonixin, Meclofenamic Acid, Mefenamic Acid, Niflumic Acid, Talniflumate, Terofenamate, Tolfenamic Acid, Aceclofenac, Acemetacin, Alclofenac, Amfenac, Amtolmetin Guacil, Bromfenac, Bufexamac, Cinmetacin, Clopirac, Diclofenac, Etodolac, Felbinac, Fenclozic Acid, Fentiazac, Glucametacin, Ibufenac, Indomethacin, Isofezolac, Isoxepac, Lonazolac, Metiazinic Acid, Mofezolac, Oxametacine, Pirazolac, Proglumetacin, Sulindac, Tiaramide, Tolmetin, Tropesin, Zomepirac, Bumadizon, Butibufen, Fenbufen, Xenbucin, Clidanac, Ketorolac, Tinoridine, Alminoprofen, Benoxaprofen, Bermoprofen, Bucloxic Acid, Carprofen, Fenoprofen, Flunoxaprofen, Flurbiprofen, Ibuprofen, Ibuproxam, Indoprofen, Ketoprofen, Loxoprofen, Naproxen, Oxaprozin, Piketoprofen, Pirprofen, Pranoprofen, Protizinic Acid, Suprofen, Tiaprofenic Acid, Ximoprofen, Zaltoprofen, Difenamizole, Epirizole, Apazone, Benzpiperylon, Feprazone, Mofebutazone, Morazone, Oxyphenbutazone, Phenylbutazone, Pipebuzone, Propyphenazone, Ramifenazone, Suxibuzone, Thiazolinobutazone, Acetaminosalol, Aspirin, Benorylate, Bromosaligenin, Calcium Acetylsalicylate, Diflunisal, Etersalate, Fendosal, Gentisic Acid, Glycol Salicylate, Imidazole Salicylate, Lysine Acetylsalicylate, Mesalamine, Morpholine Salicylate, I-Naphthyl Salicylate, Olsalazine, Parsalmide, Phenyl Acetylsalicylate, Phenyl Salicylate, Salacetamide, Salicylamide O-Acetic Acid, Salicylsulfuric Acid, Salsalate, Sulfasalazine, Ampiroxicam, Droxicam, Isoxicam, Lomoxicam, Piroxicam, Tenoxicam, epsilon-Acetamidocaproic Acid, S-Adenosylmethionine, 3-Amino-4-hydroxybutyric Acid, Amixetrine, Bendazac, Benzydamine, alpha-Bisabolol, Bucolome, Difenpiramide, Ditazol, Emorfazone, Fepradinol, Guaiazulene, Nabumetone, Nimesulide, Oxaceprol, Paranyline, Perisoxal, Proquazone, Superoxide Dismutase, Tenidap, Zileuton, 21-Acetoxypregnenolone, Alclometasone, Algestone, Amcinonide, Beclomethasone, Betamethasone, Budesonide, Chloroprednisone, Clobetasol, Clobetasone, Clocortolone, Cloprednol, Corticosterone, Cortisone, Cortivazol, Deflazacort, Desonide, Desoximetasone, Dexamethasone, Diflorasone, Diflucortolone, Difluprednate, Enoxolone, Fluazacort, Flucloronide, Flumethasone, Flunisolide, Fluocinolone Acetonide, Fluocinonide, Fluocortin Butyl, Fluocortolone, Fluorometholone, Fluperolone Acetate, Fluprednidene Acetate, Fluprednisolone, Flurandrenolide, Fluticasone Propionate, Formocortal, Halcinonide, Halobetasol Propionate, Halometasone, Halopredone Acetale, Hydrocortamate, Hydrocortisone, Loteprednol Etabonale, Mazipredone, Medrysone, Meprednisone, Methylprednisolone, Mometasone Furoate, Paramethasone, Prednicarbate, Prednisolone, Prednisolone 25-Diethylamino-acetate, Prednisolone Sodium Phosphate, Prednisone, Prednival, Prednylidene, Rimexolone, Tixocortol, Triamcinolone, Triamcinolone Acetonide, Triamcinolone Benetonide, and Triamcinolone Hexacetonide.

Compounds of the invention may be combined with one or more Beta lactamase inhibitor when used in combination with a beta-lactam class antibiotic, such as penicillin or cephalosporins. Beta-lactamase inhibitors include, but are not limited to Clavulanic acid, Sulbactam, Sultamacillin, and Tazobactam.

Compounds of the invention may also be combined with one or more efflux pump inhibitor, such as a quinazolinone efflux pump inhibitors, d-ornithine-d-homophenylalanine-3-aminoquinoline, Phe-Arg-b-naphthylamide, propafenone, a phenothiazine or thioxanthene efflux pump inhibitor, 1-aza-9-oxafluorenes, N-[4-[2-(3,4-dihydro-6,7-dimethoxy-2(1H)-isoquinolinyl)ethyl]phenyl]-9,10-dihydro-5-methoxy-9-oxo-4-Acridinecarboxamide, reserpine, Milbemycin, Cinchonine, Verapamil, L-phenylalanyl-N-2-naphthalenyl-L-Argininamide (and analogs), 5′-methoxyhydnocarpin-D, methylxanthines, FK506, a cyclosporine efflux pump inhibitor, Nocardamine and other siderophores, Amiodarone, Cyclosporin A, Ro11-2933 (DMDP), Quinidine, and the optical isomers of Propranolol, Quinine (SQ1) and Quinidine, Quinine-10,11-epoxide, Quercetin, Amitriptyline, Taxuspine C derivatives, Emodin, MC-002434; Agosterol A; Pheophorbide; pyridoquinolines such as 2,2′-[(2,8,10-trimethylpyrido[3,2-g]quinoline-4,6-diyl)bis(oxy)]bis[N,N-dimethyl-ethanamine, Gitonavir, and Gemfibrozil.

### EXAMPLES

### Abbreviations

The following abbreviations may be useful in understanding the synthetic examples described below.

### BnO Benzyloxy

DMF Dimethyl formamide
DMSO Dimethyl sulfoxide

### EtOAc Ethyl Acetate

### MeI Iodomethane

MsOH Methanesulfonic acid
RT room temperature
t-BuOK (KO^{t}Bu) Potassium tert-butoxide
TFA Trifluoroacetic acid

### THF Tetrahydrofuran

### Example 1

### Synthesis of (S)-4-((R)-3-(2-aminopropan-2-yl)pyrrolidin-1-yl)-5-fluoro-1-methyl-1H-isothiazolo[5,4-B][1,4]oxazino[2,3,4-ij]quinoline-7,8(2H,9H)-dione (Compound II)

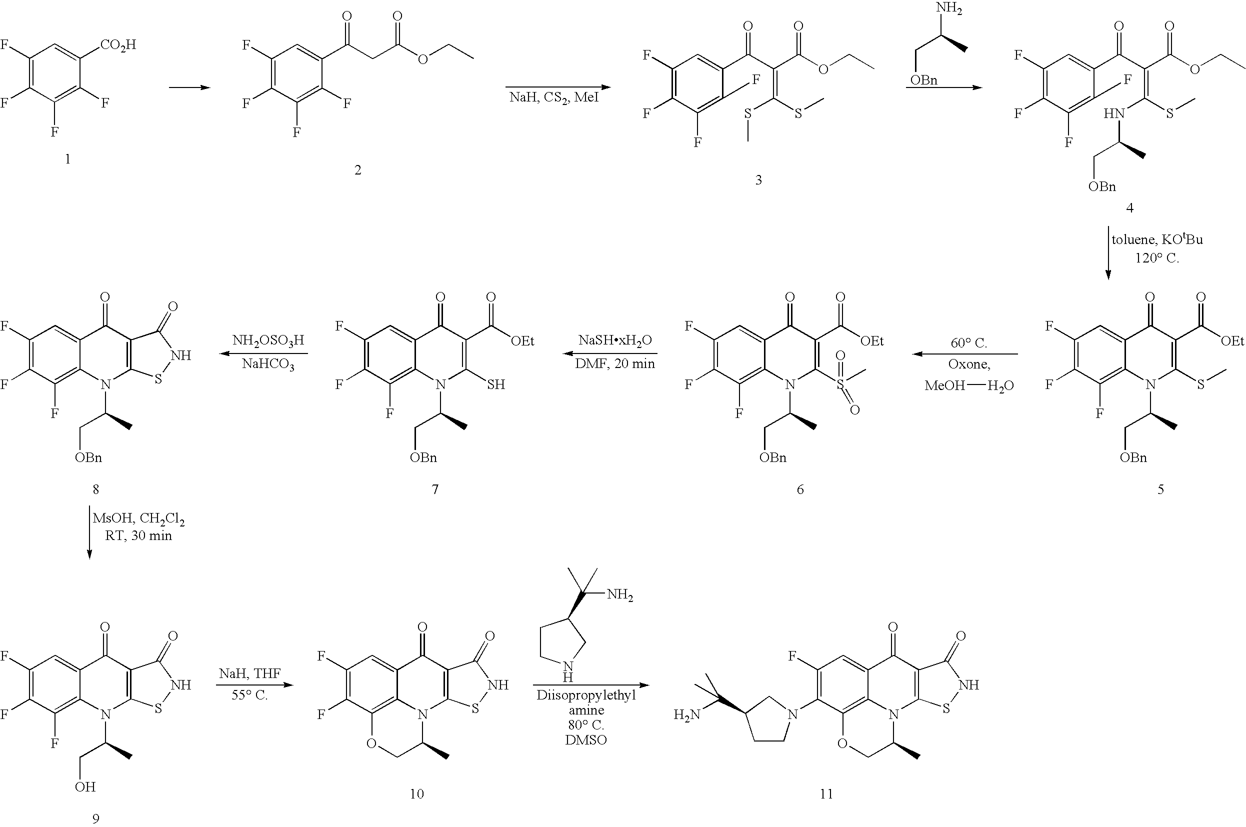

### Step 1. Preparation of Ethyl 3,3-bis(methylthio)-2-(2,3,4,5-tetrafluorobenzoyl)acrylate (compound 3)

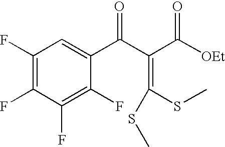

NaH (4.32 g, 60% in mineral oil, 0.11 mole) is added to an oven-dried flask and kept under Argon. 130 ml of anhydrous DMF is added to this flask. The solution is cooled to 0° C. and tetrafluoro β-keto ester 2 (13.6 g, 0.05 mole) is added carefully to the flask. The solution is stirred for 30 min at RT. The flask is again cooled to 0° C. and CS₂ (4.95 ml, 0.08 mole) is added rapidly. The reaction mixture is then stirred at RT for 25 min and 19.5 ml (0.31 mole) of MeI is added rapidly at 0° C. The resulting reaction mixture is stirred at RT for 45 min and then carefully added into 500 ml of water. The aqueous solution is extracted repeatedly with EtOAc. The combined organic layers are dried (Na₂SO₄) and concentrated. Pure 3 is obtained by silica gel flash column chromatography (eluent: 0-15% EtOAc in Hexanes) as orange yellow oil (6.4 g). ¹H-NMR (CDCl₃): δ 1.17 (3H, t, J=6.9 Hz), 2.42 (6H, s), 4.17 (2H, q, J=6.9 Hz), 7.54-7.63 (1H, m); ¹⁹F: δ −153.7 (m), −147.3 (m), −137.3 (m), −136.8 (m).

### Step 2. Preparation of (S,Z)-ethyl 3-(1-(benzyloxy)propan-2-ylamino)-3-(methylthio)-2-(2,3,4,5-tetrafluorobenzoyl)acrylate (compound 4)

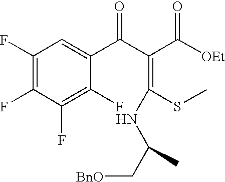

A mixture of keto ester 3 (6.4 g, 0.017 mole) and amine (2.87 g, 0.017 mole, prepared as described in Hu, X. E.; Cassady, J. M Synth. Commun. 1995, 25, 907-913) in anhydrous toluene (150 ml) is heated at 120° C. for 2 h. The reaction mixture is then cooled to RT and the solvent removed under reduced pressure. Desired compound 4 is obtained by silica gel flash column chromatography (eluent: 0-20% EtOAc in hexanes) as yellow oil (6.2 g). ¹H-NMR (CDCl₃): δ 1.00 (3H, t, J=7.2 Hz), 1.36 (3H, d, J=6.9 Hz), 2.46 (3H, s), 3.49-3.59 (2H, m), 3.99 (2H, q, J=7.2 Hz), 4.39-4.47 (1H, m), 4.56 (1H, d, J=12 Hz), 4.61 (1H, d, J=12 Hz), 7.06-7.15 (1H, m), 7.28-7.35 (5H, m), 11.62 (1H, d, J=7.8 Hz); ¹⁹F: δ −156.0 (m), −154.3 (m), −140.6 (m), −139.4 (m).

### Step. 3. Preparation of (S)-ethyl 1-(1-(benzyloxy)propan-2-yl)-6,7,8-trifluoro-2-(methylthio)-4-oxo-1,4-dihydroquinoline-3-carboxylate (compound 5)

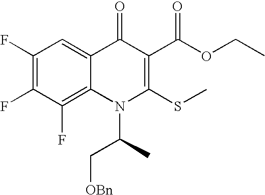

Compound 4(6.2 g, 0.013 mole) is dissolved in 100 ml of anhydrous toluene. T-BuOK (1.43 g, 0.013 mole) is added and the resulting reaction mixture heated at 120° C. for 16 h. The mixture is cooled to RT and 100 ml of water is added to the reaction mixture. The layers are separated and the aqueous layer extracted repeatedly with EtOAc. The combined organic layers are dried (Na₂SO₄) and concentrated to get 6.2 g of red oil. ¹H-NMR (CDCl₃): δ 1.40 (3H, t, J=7.2 Hz), 1.73 (3H, dd, J H—H=7.0 Hz, J H—F=3.5 Hz), 2.54 (3H, s), 3.72 (1H, ddd, J H—H=10.5, 5.0 Hz, J H—F=2.5 Hz), 3.93 (1H, ddd, J H—H=10.5, 8.5 Hz, J H—F=4.5 Hz), 4.29 (1H, d, J=11.4 Hz), 4.39 (1H, d, J=11.4 Hz), 4.40 (2H, q, J=7.2 Hz), 5.09-5.21 (1H, m), 7.05-7.09 (2H, m), 7.23-7.27 (3H, m), 7.92 (1H, ddd, J H—F=10.0, 8.5, 2.5 Hz).

### Step 4. (S)-ethyl 1-(1-(benzyloxy)propan-2-yl)-6,7,8-trifluoro-2-(methylsulfonyl)-4-oxo-1,4-dihydroquinoline-3-carboxylate (compound 6)

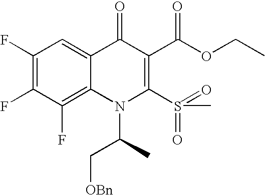

0.4 g (0.86 mmol) of the sulfide 5 is heated at 60° C. for 24 h in MeOH-water (3:1) (20 ml). The MeOH is removed under reduced pressure and the residue extracted repeatedly with EtOAc. The combined organic layers are dried (Na₂SO₄) and concentrated. The crude reaction mixture is purified by silica gel flash column chromatography (eluent: 0-50% EtOAc in Hexanes) to get 125 mg of 6 as white solid. ¹H-NMR (CDCl₃): δ 1.39 (3H, t, J=7.2 Hz), 1.70 (3H, dd, J=7.0, 1.8 Hz), 3.41 (3H, s), 3.46-3.51 (2H, m), 4.06 (1H, d, J=12.0 Hz), 4.25 (1H, d, J=12.0 Hz), 4.43 (2H, q, J=7.2 Hz), 4.87-4.94 (1H, m), 6.88-6.92 (2H, m), 7.22-7.27 (3H, m), 7.83 (1H, ddd, J=10.5, 8.1, 2.4 Hz).

Note, it is important to convert all of compound 5 to sulfone 6 because the corresponding sulfoxide (the result of incomplete oxidation of compound 5) reacts slowly with sodium hydrosulfide. The reaction time for converting compound 6 to compound 7 is also very important. If compound 6 is allowed to react with sodium hydrosulfide for an extended period of time, the C-7 fluoride group is replaced with thiol.

### Step 5. (S)-9-(1-(benzyloxy)propan-2-yl)-6,7,8-trifluoroisothiazolo[5,4-b]quinoline-3,4(2H,9H)-dione (Compound 8)

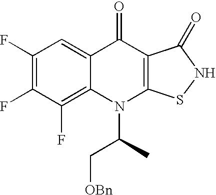

0.125 g (0.252 mmol) of 6 is dissolved in DMF (4 ml) and was degassed with Argon for 5 min. 0.042 g (0.075 mmol) of sodium hydrosulfide hydrate is added to this solution and stirred for 20 min. The solution is then diluted with water (25 ml) and acidified to pH 3 with 2N HCl. Argon is bubbled through the reaction mixture to remove H2S. The reaction mixture is then extracted repeatedly with EtOAc. The combined EtOAc layer is dried and concentrated. The resulting yellow oil 7 is then dissolved in THF-water (1:1) (6 ml). Solid NaHCO₃ is added to this solution till the pH ˜9 is reached. This solution is then cooled to 0° C. and hydroxylamine-O-sulfonic acid (0.114 g, 1 mmol) is added portion-wise. The resulting reaction mixture is stirred at RT for 3 h. The reaction mixture is made acidic (pH ˜3) with 2N aq. HCl and most of TBF is removed under reduced pressure. The residue is then extracted repeatedly with EtOAc. The combined organic layers are dried (Na₂SO₄) and concentrated to get 120 mg of 8 as thick yellow oil.

### Step 6. Preparation of (S)-4,5-difluoro-1-methyl-1H-isothiazolo[5,4-b][1,4]oxazino[2,3,4-ij]quinoline-7,8(2H,9H)-dione (Compound 10)

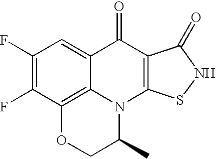

Compound 8 is taken up in 2 ml of anhydrous CH₂Cl₂. MsOH (2 ml) is added in one-portion and stirred at RT for 30 min. The CH₂Cl₂ is removed under reduced pressure. The residue is cooled to 0° C. and 10 ml of water is added carefully. Yellowish solid separates. The solid is sonicated and centrifuged. The supernatant liquid is decanted. 10 ml of water is again added to the residue. The residue is sonicated and centrifuged. Water is decanted and the solid lyophilized to get 100 mg of 9 as cream colored solid. Compound 9 is dissolved in 5 ml of anhydrous THF. The solution is cooled to 0° C. and 100 mg of NaH (excess, 60% in mineral oil) is added portion-wise. The solution is stirred at 55° C. for 24 h, cooled to 0° C. and made acidic (pH 2-3) with 2N aq HCl. Most of the THF is removed under reduced under pressure. The residue is sonicated with water (5 ml) and centrifuged. The supernatant liquid is decanted and the residue is again suspended in water (5 ml), sonicated, and centrifuged. The supernatant liquid is decanted. This is repeated 2 more times. Finally the residue is lyophilized to get 68 mg of compound 10 as cream colored solid.

1H-NMR (CDCl₃): δ 1.44 (3H, d, J=6.9 Hz), 4.37 (2H, m), 4.65 (1H, d, J=11.1 Hz), 7.71 (1H, dd, J=10.5, 8.1 Hz); ¹⁹F: δ −140.8 (d, J=24 Hz), −152.7 (d, J=24 Hz).

### Step 7. Preparation of (S)-4-((R)-3-(2-aminopropan-2-yl)pyrrolidin-1-yl)-5-fluoro-1-methyl-1H-isothiazolo[5,4-b][1,4]oxazino[2,3,4-ij]quinoline-7,8(2H,9H)-dione (Compound 11)

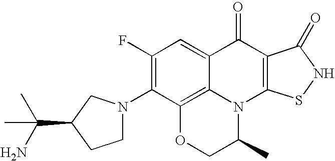

65 mg (0.21 mmol) of 10 and 50 mg of diamine are dissolved in DMSO (6 ml). The solution is degassed for 10 min with Argon. Then 56 μl of Hünig's base is added and degassed for 5 min. The reaction mixture is heated at 80° C. with stirring for 24 h. The crude reaction mixture is purified by preparative HPLC and converted from the TFA salt of 11 to the HCl salt. The solution is lyophilized to get 20 mg of compound II (HCl salt).

¹H-NMR (TFA-d): δ 1.61 (6H, d, J=6.9 Hz), 1.66 (3H, d, J=6.9 Hz), 2.36-2.62 (2H, m), 3.26-3.40 (1H, m), 4.17 (1H, t, J=11.7 Hz), 4.26-4.30 (3H, m), 4.50-4.55 (1H, m), 4.59 (1H, d, J=11.7 Hz), 4.78 (1H, d, J=12.0 Hz), 8.04 (1H, d, J=12.0 Hz); ¹⁹F (D₂O): δ −123.5.

The biological activity of compound II was tested in the assay described in Example 3. The minimal inhibitory concentration (MIC) against several bacterial strains was determined to be 0.062 for E. coli, 0.016 for methicillan sufficient S. aureus, and 0.125 for methicillan resistant S. aureus.

### Example 2

### Additional Compounds

The compounds shown in TABLE I are prepared by the methods given in Example 1. Variations to the method illustrated in Example 1 may be necessary to obtain certain compounds shown in Table I. Such variations are routine in the art of synthetic organic chemistry and are readily apparent to those of skill in the art.

TABLE I

| Cmp. | | | MIC |
|---|---|---|---|
| # | Structure | Compound Name | MRSA |
| 1 | 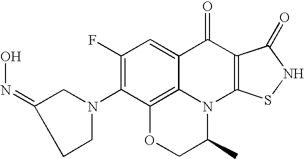 | (S,Z)-5-fluoro-4-(3-(hydroxyimino)pyrrolidin-1-yl)-1-methyl-1H-isothiazolo[5,4-b][1,4]oxazino[2,3,4-ij]quinoline-7,8(2H,9H)-dione | |
| 2 | 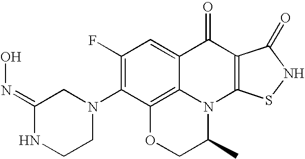 | (S,E)-5-fluoro-4-(3-(hydroxyimino)piperazin-1-yl)-1-methyl-1H-isothiazolo[5,4-b][1,4]oxazino[2,3,4-ij]quinoline-7,8(2H,9H)-dione | |
| 3 | 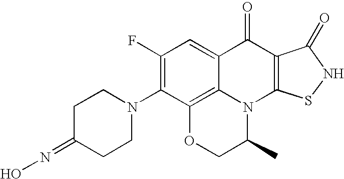 | (S)-5-fluoro-4-(4-(hydroxyimino)piperidin-1-yl)-1-methyl-1H-isothiazolo[5,4-b][1,4]oxazino[2,3,4-ij]quinoline-7,8(2H,9H)-dione | |
| 4 | 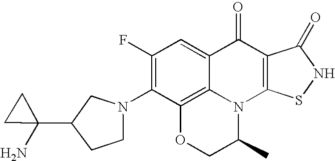 | (1S)-4-(3-(1-aminocyclopropyl)pyrrolidin-1-yl)-5-fluoro-1-methyl-1H-isothiazolo[5,4-b][1,4]oxazino[2,3,4-ij]quinoline-7,8(2H,9H)-dione | |
| 5 | 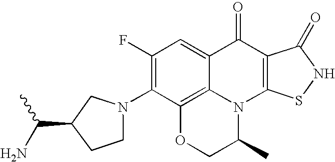 | (S)-4-((R)-3-(1-aminoethyl)pyrrolidin-1-yl)-5-fluoro-1-methyl-1H-isothiazolo[5,4-b][1,4]oxazino[2,3,4-ij]quinoline-7,8(2H,9H)-dione | |
| 6 | 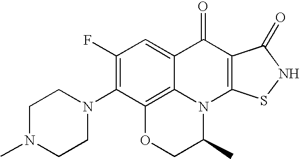 | (S)-5-fluoro-1-methyl-4-(4-methylpiperazin-1-yl)-1H-isothiazolo[5,4-b][1,4]oxazino[2,3,4-ij]quinoline-7,8(2H,9H)-dione | 4 |
| 7 | 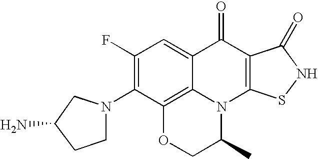 | (S)-4-((S)-3-aminopyrrolidin-1-yl)-5-fluoro-1-methyl-1H-isothiazolo[5,4-b][1,4]oxazino[2,3,4-ij]quinoline-7,8(2H,9H)-dione | 1 |
| 8 | 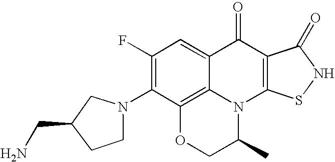 | (S)-4-((S)-3-(aminomethyl)pyrrolidin-1-yl)-5-fluoro-1-methyl-1H-isothiazolo[5,4-b][1,4]oxazino[2,3,4-ij]quinoline-7,8(2H,9H)-dione | 0.5 |
| 9 | 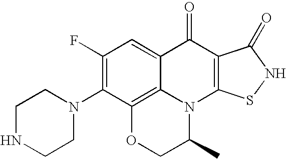 | (S)-5-fluoro-1-methyl-4-(piperazin-1-yl)-1H-isothiazolo[5,4-b][1,4]oxazino[2,3,4-ij]quinoline-7,8(2H,9H)-dione | 8 |

### Example 3

### Antimicrobial Activity of Compounds

The antimicrobial activity of the compounds of the invention may be evaluated by a number of methods, including the following visual minimum inhibitory concentration (MIC) assay. This assay determines the minimum concentration of compound required to inhibit growth of a bacterial strain.

### Minimum Inhibitory Concentration (MIC) Assay

Whole-cell antibacterial activity is determined by broth microdilution using conditions recommended by the NCCLS (see National Committee for Clinical Laboratory Standards. 2001. Performance standards for antimicrobial susceptibility testing: 11^{th} informational supplement. Vol. 21, no. 1, M100-S11. National Committee for Clinical Laboratory Standards, Wayne, Pa.). Test compounds are dissolved in DMSO and diluted 1:50 in Mueller-Hinton II broth (Becton-Dickinson) to produce a 256 μg/ml stock solution. In a 96-well microtiter plate, the compound solution is serially two-fold diluted in Mueller-Hinton II broth. After the compounds are diluted, a 50 μl aliquot of the test organism (˜1×10⁶ cfu/mL) is added to each well of the microtiter plate. The final test concentrations ranges from 0.125-128 μg/mL. Inoculated plates are incubated in ambient air at 37° C. for 18 to 24 hours. The organisms selected for testing included laboratory strains S. aureus ATCC 29213 and E. coli ATCC 25922 (strains purchased from American Type Culture Collection, Manassas, Va.), S. aureus FQR700699, and Paeruginosa 27853. The minimum inhibitory concentration (MIC) is determined as the lowest concentration of compound that inhibited visible growth of the test organism. Certain compounds of Formula I and II exhibit MIC values of less than 0.1 micromolar when evaluated in this assay against MRSA S. aureus (FQR700699), MSSA (ATCC29213), or E. coli (ATCC 25922).

### Example 4

### Cell Viability Staining with Alamar Blue

To determine whether the microcidal effect observed against S. aureus and E. coli is specific to bacterial cells, compounds are screened for cell viability effects on several human cell types.

Optimal cell density is first determined by plating cells in a 96-well plate standard sterile tissue culture plates in 100 μl media, 110% FBS at six cell densities from 500 cells/well to 15,000 cells/well. A cell free well containing only media is used as a control. Cells are incubated at 37° C. in a 5% CO2 incubator for 24 hours. 10% culture volume (10 ul) of Alamar Blue (Biosource, DAL1100, 100 mL) is then added. Cells are incubated at 37° C. in a 5% CO2 incubator and read in a Victor V plate reader, 544 nm excitation, 590 nm emission, at 3, 4, and 24 hours after the addition of Alamar Blue. The cell number vs. change in fluorescence is plotted to determine linearity of signal vs. cell number. The optimal density varies between 500-15,000 cells/well depending on the specific cell type. The optimal density is selected based on the highest number of cells that is still in the linear response range.

### Determination of Compound Cytotoxicity

Cells are plated at optimal cell density in a standard sterile tissue culture 96 well plate, and incubated at 37° C. O/N in a 5% CO2 incubator. 12 to 48 hours post-plating media is removed. The cells are washed 1 or 2 times with 1×PBS and replaced with fresh media containing the test compound in 1% DMSO. 24 to 72 hours after addition of compound, the media is removed, and the cells washed 1 to 2 times with 1×PBS. Fresh media containing 1/10 volume of Alamar Blue is then added. Plates are incubated 4 hours at 37° C. in a 5% CO2 incubator and read in a Victor V plate reader, 544 nm excitation, 590 nm emission.

Compounds are diluted to 20 micromolar in 1% DMSO and media and screened in duplicate to obtain single concentration cytotoxicity data. Eight concentration points from 0.78 micromolar to 100 micromolar, run in duplicate, are used to determine cyclotoxicity CC50 values. Cells with 1% DMSO and media are used as a negative control, compounds having a known CC50 against a particular cell type are used as positive controls.

The change in fluorescence vs. concentration of test compound is plotted to determine the cytotoxicity of the compound.

Sample media conditions, optimal plating densities, and positive control compounds for two cell types screened are presented in Table III.

Certain compounds disclosed in Examples 1 and 2 exhibit CC50 values greater than 10 uM against each of the cell lines listed below. Other cell types that may be used include but are not limited to Balb/3TC, CEM-SS, HeLa, Hep2, HepG2, HT-29, MRC-5, SK-N-SH, U-87 MG, 293T, and Huh-7. More preferred are compounds with a CC50 value greater than 50 uM. Most preferred are compounds with a CC50 value greater than 100 uM.

TABLE II

| | | | Positive |
|---|---|---|---|
| Cell Line | Media | Plating Density | Control |
| CHO | 1. F-12 Nutrient Mixture | 7,000 cells/well | Terfenadine |
| (Chinese | (Gibco #11765- 054) | | CC₅₀ = |
| hamster | containing 10% FBS, 1% | | 4.3-6.5 μM |
| ovary) | Pen Strep, 1.5 g/L | | |
| | Sodium Bicarbonate | | |
| | 2. McCoy's 5a medium, | | |
| | 10% FBS and PS/Gln | | |
| Hep 2 | Minimum Essential | 7,000 cells/well | Terfenadine |
| (laryngeal | Medium - Alpha | | CC₅₀ = |
| carcinoma) | Medium | | 3-5 μM |
| | (Gibco #12571-063) | | |
| | containing 10% FBS, | | |
| | 1% Pen Strep, 1.5 g/L | | |
| | Sodium Bicarbonate | | |

What is claimed is:

1. A compound or pharmaceutically acceptable salt of Formula I, or its tautomer of Formula II: 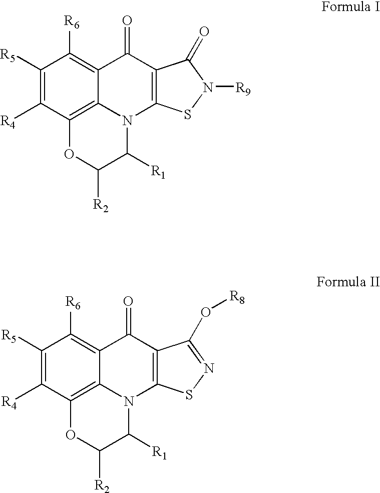
wherein:
R₁ and R₂ are independently hydrogen, halogen, amino, ═CH₂, C₁-C₄alkyl, C₂-C₄alkenyl, C₁-C₄alkoxy, mono- or di-C₁-C₄alkylamino, C₁-C₂haloalkyl, or C₁-C₂haloalkoxy;
R₄ is a nitrogen-linked heterocycloalkyl group, which has 5 or 6 ring members, including 0 or 1 additional ring heteroatoms independently chosen from N, O, and S, substituted with 0, 1, or more substituents (a), 0 or 1 substituent (b), and 1 or 2 substituents (c); where
(a) is halogen, hydroxy, amino, nitro, —C(O)NH₂, C₁-C₄alkyl, C₁-C₄alkoxy, C₁-C₂haloalkyl, or C₁-C₂haloalkoxy,
(b) is hydroxyC₁-C₄alkyl, aminoC₁-C₄alkyl, mono- or di-(C₁-C₄alkyl)amino, mono- or di-alkylcarboxamide, or phenyl, each of which is unsubstituted, and
(c) is oxo, cyano, HO—N═, C₁-C₄alkyl-O—N═, branched C₁-C₄alkyl, C₁-C₆alkylthio, C₁-C₆alkoxy, C₂-C₆alkanoyl, (C₃-C₇cycloalkyl)C₀-C₄alkyl, C₁-C₆alkylester, or an amino group of the formula or an amino group of the formula 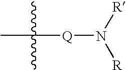
where
Q is absent or C₁-C₄alkyl,
R is hydrogen or C₁-C₄alkyl, and
R′ is (C₃-C₇cycloalkyl)(C₀-C₄alkyl), (5- or 6-membered heterocycloalkyl)C₀-C₄alkyl, or (aryl)C₀-C₄alkyl;
wherein each of (c) other than oxo, cyano, and HO—N═ is substituted with 0, 1, or 2 substituents independently chosen from halogen, hydroxy, amino, cyano, nitro, oxo, C₁-C₂alkyl, C₁-C₂alkoxy, C₁-C₂hydroxyalkyl, C₁-C₂aminoalkyl, or mono- or di-(C₁-C₂alkyl)amino;
R₅ is hydrogen, halogen, hydroxy, amino, cyano, C₁-C₄alkyl, C₁-C₄alkoxy, or mono- or di-(C₁-C₄)alkylamino;
R₆ is hydrogen, halogen, hydroxy, amino, cyano, nitro, —NHNH₂, C₁-C₄alkyl, C₁-C₄alkoxy, mono- or di-(C₁-C₄)alkylamino, mono-, di- or tri-C₁-C₄ alkylhydrazinyl, C₂-C₄alkanoyl, C₁-C₄alkylester, C₁-C₂haloalkyl, or C₁-C₂haloalkoxy;
R₅ is hydrogen, C₁-C₆alkyl, or C₂-C₆alkanoyl; and
R₉ is hydrogen, C₁-C₄alkyl, (C₃-C₇cycloalkyl)C₀-C₂alkyl, or (phenyl)C₀-C₂alkyl, each of which is substituted with 0 to 3 substituents independently chosen from halogen, hydroxy, amino, C₁-C₂alkyl, C₁-C₂alkoxy, mono- and di-(C₁-C₂)alkylamino, C₁-C₂haloalkyl, and C₁-C₂haloalkoxy.

2. A compound or salt of claim 1, wherein
R₁ is hydrogen or C₁-C₃alkyl; and
R₂ is hydrogen, ═CH₂, or C₁-C₄alkyl.

3. A compound or salt of claim 1, wherein R₁ is methyl and R₂ is hydrogen.

4. A compound or salt of claim 1, wherein R₅ is hydrogen, halogen, or amino.

5. A compound or salt of claim 1, wherein R₁ is methyl, R₂ is hydrogen, and R₅ is halogen.

6. A compound or salt of claim 1, R₆ is hydrogen, amino, C₁-C₂alkyl, mono- or di-(C₁-C₂)alkylamino.

7. A compound or salt of claim 5, wherein R₆ is hydrogen.

8. A compound or salt of claim 1, wherein the compound is a compound of Formula II and R₈ is hydrogen or the compound is a compound of Formula I and R₉ is hydrogen.

9. A compound or salt of claim 1, wherein the compound is a compound of Formula I and R₉ is C₁-C₄alkyl, (C₃-C₇cycloalkyl)C₀-C₂alkyl, or (phenyl)C₀-C₂alkyl.

10. A compound or salt of claim 2, wherein
R₅ is hydrogen, halogen, or amino;
R₆ is hydrogen, amino, C₁-C₂alkyl, mono- or di-(C₁-C₂)alkylamino;
the compound is a compound of Formula II and R₈ is hydrogen or the compound is a compound of Formula I and R₉ is hydrogen;
R₄ is a nitrogen-linked heterocycloalkyl group, in which the N-linked heterocycloalkyl group is a pyrrolidinyl, piperidinyl, piperazinyl, morpholinyl, or thiomorpholinyl group substituted with 0, 1, or more substituents (a) and 1 substituent (c); where
(a) is halogen, hydroxy, amino, —C(O)NH₂, C₁-C₂alkyl, C₁-C₂alkoxy, trifluoromethyl, or trifluoromethoxy, and
(c) is oxo, cyano, HO—N═, C₁-C₄alkyl-O—N═, branched C₁-C₄alkyl, C₁-C₄alkoxy, or (C₃-C₇cycloalkyl)C₀-C₄alkyl, wherein each of (c) other than oxo, cyano, and HO—N═ is substituted with 0, 1, or 2 substituents independently chosen from halogen, hydroxy, amino, oxo, C₁-C₂alkoxy, C₁-C₂-aminoalkyl, or mono- or di-(C₁-C₂alkyl)amino.

11. A compound of claim 10, wherein
(a) is halogen, amino, C₁-C₂alkyl, C₁-C₂alkoxy, and
(c) is HO—N═, C₁-C₂alkyl-O—N═, branched C₁-C₄alkyl, or (cyclopropyl)C₀-C₂alkyl; wherein the branched C₁-C₄alkyl and the (cyclopropyl)C₀-C₂alkyl are substituted with 0 or 1 halogen, hydroxy, amino, or mono- or di-(C₁-C₂alkyl)amino substituent.

12. A compound or salt of claim 2, wherein R₄ is a group of the formula:

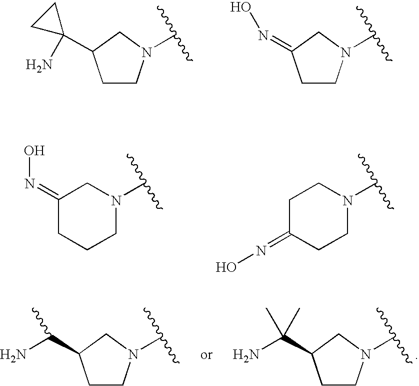

13. A compound or salt of claim 1, wherein the compound is a compound of the formula:

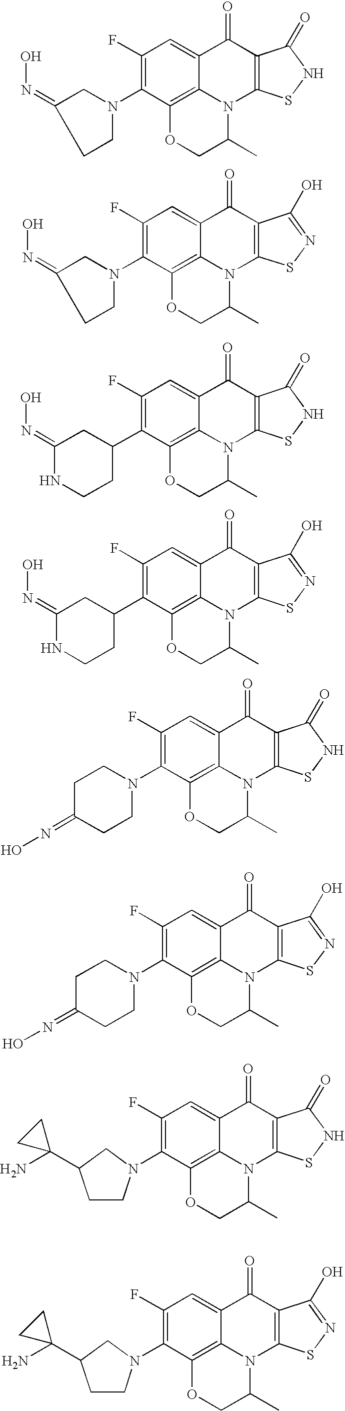

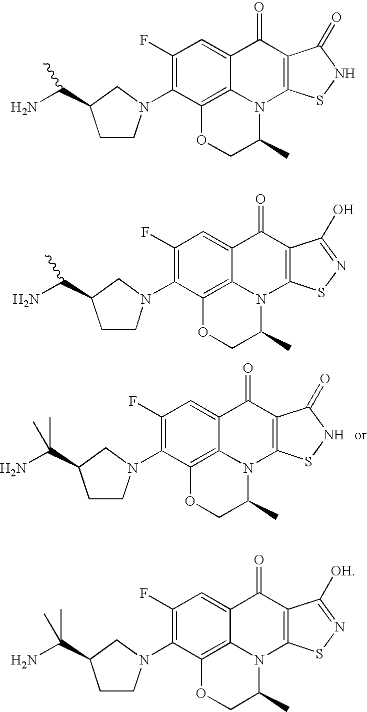

14. A pharmaceutical composition comprising a compound or salt of claim 1, together with a pharmaceutically acceptable carrier.

15. The pharmaceutical composition of claim 14, wherein the composition is formulated as an injectable fluid, an aerosol, a cream, a gel, a pill, a capsule, a tablet, a syrup, a transdermal patch, or an ophthalmic solution.

16. A composition comprising a compound or salt of claim 1 in combination with one or more other active agent, wherein the other active agent is a antibacterial agent, antiprotozoal agent, antifungal agent, antiviral agent, interferon, efflux-pump inhibitor, or beta-lactamase inhibitor.

17. A method for using the composition of claim 40 to treat a microbial infection comprising providing the composition to a patient and informing the patient that the composition can be used to treat a microbial infection.

18. The method of claim 17, wherein the informing comprises providing written instructions for using the composition to treat a patient suffering from a bacterial infection and the composition is provided in a package together with the instructions.

19. A method for treating or preventing a bacterial or protozoal infection comprising providing an effective amount of a compound or salt of claim 1 to a patient in need thereof.

20. The method of claim 19, wherein the bacterial or protozoal infection is a urinary tract infection, pyelonephritis, lower respiratory tract infection, skin infection, skin-structure infection, urethral gonococcal infection, cervical gonococcal infection, urethral chlamydial infection, cervical chlamydial infection, bone infection, joint infection, gram-negative bacterial infection, infectious diarrhea, typhoid fever, prostatitis, acute sinusitis, acute exacerbation of chronic bronchitis, pneumonia, intra-abdominal infection, gynecologic infection, or pelvic infection.

21. The method of claim 19, wherein the bacterial infection is a methicillan resistant S. aureus infection.

22. The method of claim 19, wherein the patient is a human patient.
